SEQUENCE LISTING (18 sequences)

SEQ ID NO: 1 - DNA, 50 bp, Artificial Sequence
  Decode forward primer
aatgatacggcgaccaccgagatctacaccggtgcctgagtttgtttgaa

SEQ ID NO: 2 - DNA, 46 bp, Artificial Sequence
  Decode reverse primer
caagcagaagacggcatacgagatggcattaaagcagcgtatccac

SEQ ID NO: 3 - DNA, 38 bp, Artificial Sequence
  Decode Illumina sequencing primer
gaaggctcgagaaggtatattgctgttgacagtgagcg

SEQ ID NO: 4 - DNA, 67 bp, Artificial Sequence
  Sequencing primer
cggtgccactttttcaagttgataacggactagccttattttaacttgctatttctagctctaaaac

SEQ ID NO: 5 - DNA, 71 bp, Artificial Sequence
  Indexing primer
tttcaagttacggtaagcatatgatagtccattttaaaacataattttaaaactgcaaactacccaagaaa

SEQ ID NO: 6 - DNA, 47 bp, Artificial Sequence
  Reverse primer
aatgatacggcgaccaccgagatctacaccgactcggtgccactttt

SEQ ID NO: 7 - DNA, 54 bp, Artificial Sequence
  Forward primer 1
caagcagaagacggcatacgagatcatcacgtttcttgggtagtttgcagtttt

SEQ ID NO: 8 - DNA, 54 bp, Artificial Sequence
  Forward primer 2
caagcagaagacggcatacgagatccgatgttttcttgggtagtttgcagtttt

SEQ ID NO: 9 - DNA, 54 bp, Artificial Sequence
  Forward primer 3
caagcagaagacggcatacgagatcttaggctttcttgggtagtttgcagtttt

SEQ ID NO: 10 - DNA, 54 bp, Artificial Sequence
  Forward primer 4
caagcagaagacggcatacgagatctgaccatttcttgggtagtttgcagtttt

SEQ ID NO: 11 - DNA, 54 bp, Artificial Sequence
  Forward primer 5
caagcagaagacggcatacgagatcacagtgtttcttgggtagtttgcagtttt

SEQ ID NO: 12 - DNA, 54 bp, Artificial Sequence
  Forward primer 6
caagcagaagacggcatacgagatcgccaattttcttgggtagtttgcagtttt

SEQ ID NO: 13 - DNA, 54 bp, Artificial Sequence
  Forward primer 7
caagcagaagacggcatacgagatccagatctttcttgggtagtttgcagtttt

SEQ ID NO: 14 - DNA, 54 bp, Artificial Sequence
  Forward primer 8
caagcagaagacggcatacgagatcacttgatttcttgggtagtttgcagtttt

SEQ ID NO: 15 - DNA, 54 bp, Artificial Sequence
  Forward primer 9
caagcagaagacggcatacgagatcgatcagtttcttgggtagtttgcagtttt

SEQ ID NO: 16 - DNA, 54 bp, Artificial Sequence
  Forward primer 10
caagcagaagacggcatacgagatctagctttttcttgggtagtttgcagtttt

SEQ ID NO: 17 - DNA, 54 bp, Artificial Sequence
  Forward primer 11
caagcagaagacggcatacgagatcggctactttcttgggtagtttgcagtttt

SEQ ID NO: 18 - DNA, 54 bp, Artificial Sequence
  Forward primer 12
caagcagaagacggcatacgagatccttgtatttcttgggtagtttgcagtttt

(19) United States
(12) Patent Application Publication
(10) Pub. No.: US 2020/0078387 A1
(43) Pub. Date: Mar. 12, 2020

(54) CHEMICAL INHIBITORS AGAINST KINASES TO BLOCK TELOMERE ELONGATION IN CANCER

(71) Applicant: THE JOHNS HOPKINS UNIVERSITY, Baltimore, MD (US)

(72) Inventors: Carol W. Greider, Baltimore, MD (US); Steven Wang, Baltimore, MD (US)

(21) Appl. No.: 16/469,615

(22) PCT Filed: Dec. 15, 2017
(86) PCT No.: PCT/US2017/066674

§ 371 Date: Jun. 13, 2019

Related U.S. Application Data
(60) Provisional application No. 62/435,191, filed on Dec. 16, 2016.

(51) Int. Cl. A61K 31/7076; G01N 33/50; A61K 31/4439; A61K 31/4045; A61K 31/5377; A61K 31/551; A61K 31/655; A61K 31/4745; A61K 31/5025; C12Q 1/48; A61K 31/4725; A61K 31/519

(52) U.S. Cl. CPC A61K 31/7076; G01N 33/5011; A61K 31/4439; A61K 31/4045; A61K 31/5377; A61K 31/519; A61K 31/655; A61K 31/4745; A61K 31/5025; C12Q 1/485; A61K 31/4725; A61K 31/551

(57) ABSTRACT

The present invention relates generally to chromosome maintenance and cell viability, and more specifically, to the identification of kinases the inhibition of which blocks telomere elongation. In one aspect, the present invention provides methods for treating cancer. The methods generally comprise the administration of agents that interfere with the lengthening of telomeres in cancer. In a specific embodiment, a method of treating cancer in a subject by interfering with lengthening of telomeres in cancer cells comprises administering to the cells an effective amount of an inhibitor of casein kinase 1 (CK1), wherein the administration of the inhibitor leads to progressive telomere shortening in the cancer cell, thereby treating cancer in the subject. In another embodiment, the method can further comprise administering to the cells an effective amount of an inhibitor of bromodomain-containing protein 4 (BRD4) and/or an inhibitor of the MEK/ERK pathway.

### CROSS-REFERENCE TO RELATED APPLICATIONS

This application claims the benefit of U.S. Provisional Application No. 62/435,191, filed Dec. 16, 2016, which is incorporated herein by reference in its entirety.

### FIELD OF THE INVENTION

The present invention relates generally to chromosome maintenance and cell viability, and more specifically, to the identification of kinases the inhibition of which blocks telomere elongation.

### INCORPORATION-BY-REFERENCE OF MATERIAL SUBMITTED ELECTRONICALLY

This application contains a sequence listing. It has been submitted electronically via EFS-Web as an ASCII text file entitled “P14464-02_ST25.txt.” The sequence listing is 3,855 bytes in size, and was created on Dec. 15, 2017. It is hereby incorporated by reference in its entirety.

### BACKGROUND OF THE INVENTION

Replicative immortality is a hallmark of cancer. Most adult mammalian cells do not express telomerase, and will eventually undergo replicative senescence as telomeres shorten. One of the most common ways cancer cells overcome this is by activating telomerase expression. Telomerase is able to maintain telomeres such that they are not recognized as DNA damage, and the cell is able to divide indefinitely. Thus, developing telomerase inhibitors, or inhibitors of telomere elongation, are an area of significant investigation.

### SUMMARY OF THE INVENTION

The present invention is based, at least in part, on the identification of BRD4, CK1 and the MEK/ERK pathway as positive regulators of telomere length. Identification of these pathways generates new potential targets for blocking telomere elongation in cancer.

In one aspect, the present invention provides methods for treating cancer. The methods generally comprise the administration of agents that interfere with the lengthening of telomeres in cancer. In a specific embodiment, a method of treating cancer in a subject by interfering with lengthening of telomeres in cancer cells comprises administering to the cells an effective amount of an inhibitor of casein kinase 1 (CK1), wherein the administration of the inhibitor leads to progressive telomere shortening in the cancer cell, thereby treating cancer in the subject. CK1 inhibitors useful in the methods of the present invention include, but are not limited to, D4476, PF670462, IC261, PF4800567, PF5006739, SR-3029, CKI dihydrochloride, (R)-CR8, ®-DRF053 dyhydrochloride, LH846, TA 01, TA 02, TAK 715, A-3 hydrochloride, and IC261. Other CK1 inhibitors are described herein.

Any type of cancer can be treated using the methods and compositions described herein. Such cancers include, but are not limited to, stomach cancer, osteosarcoma, lung cancer, pancreatic cancer, adrenocortical carcinoma, melanoma, breast cancer, ovarian cancer, cervical cancer, skin cancer, connective tissue cancer, uterine cancer, anogenital cancer, central nervous system cancers, retinal cancer, blood and lymphoid cancers, kidney cancer, bladder cancer, colon cancer and prostate cancer.

In particular embodiments, the inhibitor is a small molecule, a peptide, a nucleic acid molecule, or a protein. In certain embodiments, the nucleic acid molecule is an siRNA, shRNA, miRNA, Locked Nucleic Acid (LNA), antisense oligonucleotide, a chemically modified oligonucleotide, or a combination thereof.

In another embodiment, the method can further comprise administering to the cells an effective amount of an inhibitor of bromodomain-containing protein 4 (BRD4) and/or an inhibitor of the MEK/ERK pathway. In particular embodiments, the BRD4 inhibitor inhibits the kinase activity of BRD4. Specific BRD4 inhibitors useful in the methods of the present invention include, but are not limited to, JQ1, PFI-1, RVX-208 (Resverlogix Corp.), I-BET-762 (GlaxoSmithKline), I-BET-151 (GlaxoSmithKline), CPI-203, BMS-986158, OTX015, PLX-51107, GSK525762, GSK2820151, and BAY1238097. Other BRD4 inhibitors are described herein. MEK/ERK inhibitors include, but are not limited to, BRAF inhibitors (vemurafenib, dabrafenib, LGX818), MEK inhibitors (trametinib, selumetinib, MEK162, PD98059, U0126, CI-1040 (PD184352), PD325901, AZD6244) and MAPK/ERK inhibitors. MAPK/ERK inhibitors include, but are not limited to, RO4402257 (Hoffman-LaRoche), RO3201195 (Hoffman-LaRoche), PH-797804 (Pfizer), AZD-6703 (AstraZeneca), GW681323 (GlaxoSmithKline), VX-745 (Vertex Pharmaceuticals), VX-702 (Vertex Pharmaceuticals), Scio-469 (Scios/Johnson & Johnson), Scio-323 (Scios/Johnson & Johnson), TAK-715 (Takeda Pharmaceuticals), RWJ-67657 (Johnson & Johnson), BIRB-796 (Boehringer Ingelheim), KC706 (Kemia), ARRY-797 (Array BioPharma), AMG-548 (Amgen), CC-401 (Celgene), SP600125 (Celgene), CEP-1347 (Cephalon), Sorafenib Bayer/Onyx), Raf265 (Novartis), PLX4032 (Roche/Plexxikon), PD0325901 (Pfizer), ARRY-142886 (Array BioPharma), AZD6244 (AstraZeneca/Array Biopharma), ARRY-438162 (Array BioPharma), RDEA119/BAY-869766 (Ardea Biosciences/Bayer), and RDEA436 (Ardea Biosciences). Other MEK/ERK inhibitors are described herein. In yet another embodiment, the method can further comprise administering a chemotherapeutic agent. The method can also comprise administering a cancer immunotherapy drug. In particular embodiments, the subject is human. In particular embodiments, the inhibitors of CK1, BRD4 and MEK/ERK inhibit the kinase activity of such proteins. Additional targets include those described in Table 3.

In another specific embodiment, the method further comprises measuring telomere length. Telomere length can be measured prior to and after contacting with the inhibitor. In certain embodiments, the measuring step comprises flow-FISH (flow cytometry with fluorescent in situ hybridization. In another embodiment, the measuring step comprises a Southern blot. In another embodiment, the measuring step comprises a technique that includes PCR. For example, the technique can be a modified single telomere length analysis (STELA). In a further embodiment, the method further comprises nucleic acid sequencing.

In another aspect, the present invention provides methods for increasing telomere length. In one embodiment, a method of increasing telomere length comprises contacting a cell with an agent that activates BRD4, CK1, MEK/ERK pathway or a combination thereof, thereby elongating telomeres in the cell. In particular embodiments, the agent is a small molecule, a peptide, a nucleic acid molecule, or a protein. The nucleic acid molecule can be an siRNA, shRNA, miRNA, Locked Nucleic Acid (LNA), antisense oligonucleotide, a chemically modified oligonucleotide, or a combination thereof.

In a specific embodiment, the agent increases expression or activity of BRD4. In another embodiment, the agent increases expression or activity of CK1. In yet another embodiment, the agent is an activator of the MEK/ERK pathway. In certain embodiments, the cell is a mammalian cell. In more particular embodiments, the cell is a human or murine cell. In one embodiment, telomere length is increased at least 50% as compared to telomere length prior to contacting with the agent.

The present invention also provides a method of treating a telomere syndrome in a subject, wherein the syndrome is characterized by shortened telomere length, the method comprising administering to the subject a BRD4 agonist, a CK1 agonist, MEK/ERK pathway agonist or a combination thereof, wherein administration of the agonist leads to progressive telomere lengthening, thereby treating the telomere syndrome in the subject. A telomere syndrome can include, but is not limited to, dyskeritosis congenita, bone marrow failure, aplastic anemia, and pulmonary fibrosis.

In particular embodiments, the agent is a small molecule, a peptide, a nucleic acid molecule, or a protein. In a specific embodiment, the CK1 agonist is pyrvinium pamoate. In certain embodiments, the subject is human. In one embodiment, telomere length is increased at least 50% as compared to telomere length prior to contacting with the agonist. In certain embodiments, the agonist can be a small molecule, peptide, siRNA, etc. against inhibitors of BRD4, CK1 and/or MEK/ERK.

In another specific embodiment, the method further comprises measuring telomere length. Telomere length can be measured prior to and after contacting with the inhibitor. In certain embodiments, the measuring step comprises flow-FISH (flow cytometry with fluorescent in situ hybridization. In another embodiment, the measuring step comprises a Southern blot. In one embodiment, the measuring step comprises a technique that includes PCR. For example, the technique can be a modified single telomere length analysis (STELA). In a further embodiment, the method further comprises nucleic acid sequencing.

### BRIEF DESCRIPTION OF THE FIGURES

FIG. 1A-1D. Selecting cell line to perform pooled telomere length screen. FIG. 1A: Transduction efficiency of indicated cell lines, measured by percent GFP+ after 72 post transduction with FUGW at MOI=1. FIG. 1B: Telomere length of indicated cell lines, measured by telomere flow-FISH. Bovine thymocyte had a known telomere length and served as a control for telomere length. FIG. 1C: Flow cytometry plot of cell cycle profile of K-562, 293FT and Hela, following staining with PI. FIG. 1D: Telomere length of K-562, 293FT and HeLa, measured by flow-FISH.

FIG. 2A-2C. Knockdown of hTERT or POT1 shortens, or elongates telomeres respectively, in 293FT cells. FIG. 2A: Q-PCR of POT1 or hTERT levels, following shRNA knockdown, in 293FT cells. FIG. 2B: Telomere length of 293FT cells following hTERT or POT1 knockdown, measured by flow-FISH. FIG. 2C: Overlay of flow-FISH telomere signals of hTERT, non-silencing and POT1 shRNA.

FIG. 3A-3D. Optimizations for fluorescence activated cell sorting and flow-FISH. Adaptations to the traditional telomere flow-FISH protocol were made to enhance DNA staining and cell cycle profile (FIG. 3A) and decrease aggregation to increase cell sorting speed and accuracy. FIG. 3A: PI staining of HeLa cells fixed with 70% ethanol. FIG. 3B: PI staining of HeLa cells fixed with paraformaldehyde and methanol. FIG. 3C: Flow cytometry of PI signal for HeLa cells stained with traditional PI staining solution (Pulse width vs Area, circled population represents singlets). FIG. 3D: Flow cytometry of PI signal for HeLa cells stained with 0.1% SDS PI staining solution (Pulse width vs Area, circled population represents singlets).

FIG. 4. POT1 and hTERT shRNA inserts are recovered in the expected fractions upon sorting long and short telomere cells. The percentage of the short fraction, unsorted fraction or long fraction that contained hTERT, non-silencing, or POT1 inserts. hTERT reads are enriched in the short fraction because loss of hTERT causes short telomeres, which enriches the short fraction for cells with hTERT reads. POT1 reads are enriched in the long fraction because loss of POT1 causes long telomeres, which enriches the long fraction for cells with POT1 reads.

FIG. 5A-5C. Inhibition of BRD4 with small molecules blocks SVA mediated telomere elongation. Telomeres were rapidly elongated in 6 days, after transduction with SVA, a lentivirus that expresses telomerase. Cells were treated chemical inhibitors, to determine the effect of the inhibitor on telomere elongation. FIG. 5A: Southern blot of telomere length at days 2 and 6 post SVA transduction, in the presence of DMSO, 10 pLM KU-55933 (an ATM inhibitor), or 0.1 μM JQ1 (a BRD4 inhibitor). FIG. 5B: Southern blot of telomere length at days 2 and 6 post SVA transduction, in the presence of DMSO or 0.4 μM OTX015 (a BRD4 inhibitor). FIG. 5C: Southern blot of telomere length at days 2 and 6 post SVA transduction, in the presence of DMSO, 1 μM I-BET151 or 5 μM MS436 (both BRD4 inhibitors).

FIG. 6. Inhibition of BRD4 by JQ1 blocks telomere elongation in a dose dependent manner. Cells were transduced with SVA and either DMSO, 10 μM KU-55933, 100 nM JQ1, 33 nM JQ1, or 11 nM JQ1. Telomere length was measured at days 2 and 6 post-transduction by telomere Southern blot.

FIG. 7A-7B. Inhibition of CK1 by D4476 inhibits telomere elongation in a dose dependent manner. Telomeres were rapidly elongated in 6 days, after transduction with SVA, a lentivirus that expresses telomerase. Cells were treated with chemical inhibitors, to determine the effect of the inhibitor on telomere elongation. FIG. 7A: Southern blot of telomere length at days 2 and 6 post SVA transduction, in the presence of DMSO, 10 μM D4476 (a CK1 inhibitor), 0.5 μM 5-Iodotubercidin (a CK1 inhibitor), or 0.5 μM IC261 (a CK1 inhibitor). FIG. 7B: Southern blot of telomere length at days 2 and 6 post SVA transduction, in the presence of DMSO or 3.33 μM, 1.11 μM or 0.37 μM D4476.

FIG. 8A-8B. Chemical inhibition of ERK1/2 or MEK1/2 partially blocks telomere elongation. Telomeres were rapidly elongated in 6 days, following transduction with SVA, a lentivirus that expresses telomerase. Cells were treated with chemical inhibitors, to determine the effect of the inhibitor on telomere elongation. FIG. 8A: Southern blot of telomere length at days 2 and 6 post SVA transduction, in the presence of DMSO, 10 μM GDC-0994 (an ERK 1/2 inhibitor), or 10 FR-180204 (an ERK1/2 inhibitor). FIG. 8B: Southern blot of telomere length at days 2 and 6 post SVA transduction, in the presence of DMSO, 10 nM trametinib (a MEK1/2), or 5 μM selumetinib (a MEK1/2 and ERK1/2 inhibitor).

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Mutation(s) in both core component of telomerase as well as the accessory factors can cause critically short telomeres, dysfunctional telomeres, leading to age-related degenerative disorders. The first such mutation found in dyskerin, which is one of the critical accessory factors for human telomerase, causes a rare disorder called X-linked dyskeratosis congenita. Later it was found the mutations in human TR underlie bone marrow failure in autosomal dominant dyskeratosis congenita. Further studies identified additional mutations in telomerase components and telomere-related genes that lead to dysfunctional telomeres in autosomal dominant disease. It is now well characterized that short telomeres have a causal role in a broad spectrum of degenerative disorders called as “telomere syndromes,” including idiopathic pulmonary fibrosis, bone marrow failure, and other premature aging disorders. These disorders are primarily caused by defects that lead to short telomeres.

Short telomeres induce a DNA damage response, senescence and apoptosis; thus, maintaining telomere length equilibrium is essential for cell viability. In humans, syndromes of telomere shortening cause age-related degenerative diseases including dyskeratosis congenita, pulmonary fibrosis, aplastic anemia and others. The present invention may provide a novel approach to manipulate telomere length by using a BRD4 agonist, a CK1 agonist (e.g., pyrvinium pamoate), a MEK/MAPK agonist or combinations thereof. In certain embodiments, the agonist can be a small molecule, peptide, siRNA, etc. against inhibitors of BRD4, CK1 and/or MEK/ERK.

Accordingly, the present invention provides a method of treating telomere syndrome in a subject. The method includes administering to the subject a BRD4 agonist, a CK1 agonist and/or a MEK/MAPK agonist, wherein administration leads to progressive telomere lengthening, thereby treating the telomere syndrome in the subject. In one aspect, the syndrome is selected from the telomere syndromes including, dyskeritosis congenita, bone marrow failure, aplastic anemia, and pulmonary fibrosis. The methods could further include PARP1 inhibitor, an activator of Cdkl, or a combination thereof.

On the other hand, cancer cells divide continuously and increase or maintain telomere lengths to avoid cell death. The majority of human cancers maintain telomere lengths via up-regulated telomerase activity or activation of the alternative lengthening of telomeres (ALT) pathway. Recent studies associate certain mutations in TERT promoter and telomere binding proteins, such as POT1, with predisposition to cancer.

As such, in another aspect, the present invention provides a method of treating cancer in a subject by interfering with lengthening of telomeres in cancer cells. The method includes administering to the cells an effective amount of an inhibitor of a regulator of telomere lengthening, wherein the administration of the inhibitor leads to progressive telomere shortening in the cancer cells, thereby treating cancer in the subject.

In one aspect, the methods of the invention may further include measuring telomere length. In certain embodiments, the measuring step comprises flow-FISH (flow cytometry with fluorescent in situ hybridization. In another embodiment, the measuring step comprises a Southern blot. Measuring can also be accomplished by a technique that includes PCR, such as a modified single telomere length analysis (STELA) or by PCR followed by nucleotide sequencing. STELA was developed in 2003 by Duncan Baird. This technique allows investigations that can target specific telomere ends, which is not possible with TRF analysis described below.

Several techniques may be employed to assess average telomere length in eukaryotic cells. The most widely used method is the Terminal Restriction Fragment (TRF) Southern blot, which involves hybridization of a radioactive ³²P-(TTAGGG)n oligonucleotide probe to restriction enzyme digested genomic DNA embedded on a nylon membrane and subsequently exposed to autoradiographic film or phosphoimager screen. Another histochemical method, termed Q-FISH, involves fluorescent in situ hybridization (FISH).

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, as well as primates (including monkeys, chimpanzees, orangutans and gorillas) are included within the definition of subject.

The terms “administration” or “administering” are defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The route of administration of a composition containing an agent as identified herein, will depend, in part, on the chemical structure of the molecule. Polypeptides and polynucleotides, for example, are not particularly useful when administered orally because they can be degraded in the digestive tract. However, methods for chemically modifying polynucleotides and polypeptides, for example, to render them less susceptible to degradation by endogenous nucleases or proteases, respectively, or more absorbable through the alimentary tract are well known. For example, a peptide agent can be prepared using D-amino acids, or can contain one or more domains based on peptidomimetics, which are organic molecules that mimic the structure of peptide domain; or based on a peptoid such as a vinylogous peptoid. Where the agent is a small organic molecule, it can be administered in a form that releases the active agent at the desired position in the body (e.g., the stomach), or by injection into a blood vessel that the agent circulates to the target cells.

Regardless of the route of administration selected, the compounds of the present invention, which may be used in a suitable hydrated form, and/or the pharmaceutical compositions of the present invention, are formulated into pharmaceutically acceptable dosage forms such as described herein or by other conventional methods known to those of skill in the art.

The total amount of an agent to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of agent depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

In general, a suitable daily dose of a compound of the invention will be that amount of the compound which is the lowest dose effective to produce a therapeutic effect. Such an effective dose will generally depend upon the factors described above. Generally, intravenous and subcutaneous doses of the compounds of this invention for a patient will range from about 0.0001 to about 100 mg per kilogram of body weight per day which can be administered in single or multiple doses.

A “therapeutically effective amount” or “efficacious amount” means the amount of a compound that, when administered to a mammal or other subject for treating a disease, condition, or disorder, is sufficient to effect such treatment for the disease, condition, or disorder. The “therapeutically effective amount” will vary depending on the compound, the disease and its severity and the age, weight, and the like, of the subject to be treated. Typically a therapeutically effective amount should produce a serum concentration of drug of from about 0.1 ng/ml to about 50-100 ag/ml. In various embodiments, the dosage administered is sufficient to result in a serum concentration level of the drug in the subject of greater than about 0.5, 1, 2, 3, 4, 5, 6, 7, 8, 9, 10, 11, 12, 13, 14, 15, 16, 17, 18, 19, 20, 25 or 30 jM. The pharmaceutical compositions typically should provide a dosage of from about 0.001 mg to about 2000 mg of compound per kilogram of body weight per day. The drug may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If desired, the effective daily dose of the active compound may be administered as two, three, four, five, six or more sub-doses administered separately at appropriate intervals throughout the day, optionally, in unit dosage forms. There may be a period of no administration followed by another regimen of administration.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

Additionally, in various aspects, a physician or veterinarian having ordinary skill in the art can readily determine an appropriate subject for administration of the compounds described herein. For example, one of skill in the art is capable of routine diagnosis of diabetes. Also, it is routine for one of skill in the art to determine the appropriate compounds to be administered to the subject as well as the timing of administration depending on the diagnosis. Additionally, it may be appropriate to administer the compound in combination with other drugs, such as chemotherapeutic agents. When other therapeutic agents are employed in combination with the compounds of the present invention they may be used for example in amounts as noted in the Physician Desk Reference (PDR) or as otherwise determined by one having ordinary skill in the art.

In certain embodiments, pharmaceutical compositions may include, for example, at least about 0.01 mg/g of body weight of a drug, such as an inhibitor. In other embodiments, the drug may comprise between about 0.1% to about 75% of the weight of the unit, or between about 2% to about 20%, for example, and any range derivable therein. In other non-limiting examples, a dose may also comprise from about 1 jag/kg/body weight, about 100 jag/kg/body weight, about 500 jag/kg/body weight, about 1 mg/kg/body weight, about 5 mg/kg/body weight, about 10 mg/kg/body weight, about 50 mg/kg/body weight, about 100 mg/kg/body weight, about 200 mg/kg/body weight, about 300 mg/kg/body weight, about 350 mg/kg/body weight, about 400 mg/kg/body weight, about 450 mg/kg/body weight, about 500 mg/kg/body weight, about 600 mg/kg/body weight, about 700 mg/kg/body weight, about 800 mg/kg/body weight, about 900 mg/kg/body weight, about 1000 mg/kg/body weight, about 2000 mg/kg/body weight to about 5000 mg/kg/body weight or more per administration, and any range derivable therein. In non-limiting examples of a derivable range from the numbers listed herein, a range of about 350 mg/kg/body weight to about 1000 mg/kg/body weight, about 50 jag/kg/body weight to about 500 mg/kg/body weight, and the like, can be administered.

The present invention describes agents, such as chemical compounds or nucleic acid molecules that modulate de novo telomere addition by affecting a gene or pathway implicated in telomere extension via telomerase, such as a kinase pathway, for example, the BRD4 or CK1 kinase pathway or a MEK/ERK pathway.

As used herein, an agent identified as a regulator of telomere length acts to increase extension of telomeres or inhibit telomere extension. The agent may interact directly with a gene promoter to effectuate an increase or decrease in transcription or the agent may interact in a number of other ways to indirectly increase telomere addition. For example, the agent may activate a particular signal transduction pathway leading to increased or decreased transcription of a gene. Alternatively, the agent may act to suppress repressors of transcription by direct binding to the transcriptional repressor thus blocking binding of the repressor to a promoter. Alternatively, the agent may act indirectly to suppress transcriptional repressors or increase transcription.

Specific BRD4 inhibitors useful in the methods of the present invention include, but are not limited to, JQ1, PFI-1, RVX-208 (Resverlogix Corp.), I-BET-762 (GlaxoSmithKline), I-BET-151 (GlaxoSmithKline), CPI-203, BMS-986158, OTX015, PLX-51107, GSK525762, GSK2820151, and BAY1238097.

Other BRD4 inhibitors include inhibitors developed by Gilead Sciences, Inc. (U.S. Patent Applications Publication No. 2016/0075695; Publication No. 20160031868; Publication No. 20150148344; Publication No. 2014/0336190; Publication No. 2014/0296246); Dana Farber Cancer Institute (U.S. Patent Applications Publication No. 2016/0243247; Publication No. 2016/0235731; Publication No. 2013/0252331); Memorial Sloan Kettering Cancer Center (U.S. Patent Application Publication No. 2015/0342960); Bristol-Myers Squibb Company (U.S. Patent Applications Publication No. 2016/0326173; Publication No. 2016/0176864); University of Southern California (U.S. Patent Applications Publication No. 2016/0136146); Board of Regents, The University of Texas System (U.S. Patent Applications Publication No. 2016/0060260); Constellation Pharmaceuticals, Inc. (U.S. Patent Application Publication No. 2014/0296243).

CK1 inhibitors useful in the methods of the present invention include, but are not limited to, D4476, PF670462, IC261, PF4800567, PF5006739, SR-3029, CKI dihydrochloride, (R)-CR8, ®-DRF053 dyhydrochloride, LH846, TA 01, TA 02, TAK 715, A-3 hydrochloride, and IC261. Other CKI1 inhibitors include compounds developed by Proteome Sciences, Inc. (including PS110 and PS278-05) (U.S. Pat. No. 8,822,171 and U.S. Patent Applications Publication No. 2013/0123133 and Publication No. 2015/0051097); The Scripps Research Institute) (including SR-653234, SR-1277, SR-2890, SR-3029) (U.S. Pat. No. 9,493,464); and C.N.R.S. (including 4-(2-amino-4-oxo-2-imidazolin-5-ylidene)-4,5,6,7-tetrahydropyrrolo (2,3-c) azepine-8-one and 4-(2-amino-4-oxo-2-imidazolin-5-ylidene)-2-bromo-4,5,6,7-tetrahydropyrrolo (2,3-c) azepine-8-one) ((U.S. Pat. No. 7,098,204)

Other CK1 inhibitors include compounds developed by Electrophoretics Limited (U.S. Patent Application Publication No. 2016/0354375). Such compounds include, but are not limited to, 2-Phenyl-N-(pyridin-4-yl)quinazolin-4-amine (Compound 700); N-(2,3-Dihydro-1,4-benzodioxin-6-yl)-2-phenylquinazolin-4-amine (Compound 868); 4-{[2-(Thiophen-3-yl)quinazolin-4-yl]amino)}benzamide (Compound 870); 2-Phenyl-N-(1H-pyrazol-4-yl)quinazolin-4-amine (Compound 894); 2[2-Methyl-4-(pyridin-4-yl)pyrimidin-5-yl]-1,3-benzoxazole (Compound 949); 2-[2-(Pyridin-2-yl)-4-(pyridin-4-yl)pyrimidin-5-yl]-1,3-benzoxazole (Compound 950); 5-(1,3-Benzoxazol-2-yl)-2-oxo-6-(pyridin-4-yl)-1,2-dihydropyridine-3-carbonitrile (Compound 951); 4-[(2-Phenylquinazolin-4-yl)amino]phenol (Compound 957); N-(4-Methoxyphenyl)-2-(thiophen-2-yl)quinazolin-4-amine (Compound 973); N-(4-Methoxyphenyl)-2-(pyridin-2-yl)quinazolin-4-amine (Compound 977); 6-Phenyl-2-(pyridin-2-yl)-N-(pyridin-4-yl)pyrimidin-4-amine (Compound 986); 4-[(2-Phenylquinazolin-4-yl)amino]benzamide (Compound 993); 2-Phenyl-N-(pyridin-4-yl)quinazolin-4-amine (Compound 700); 2-Phenyl-N-(1H-pyrazol-4-yl)quinazolin-4-amine (Compound 894); 2-[2-Methyl-4-(pyridin-4-yl)pyrimidin-5-yl]-1,3-benzoxazole (Compound 949); 6-Phenyl-2-(pyridin-2-yl)-N-(pyridin-4-yl)pyrimidin-4-amine (Compound 986); 2-Phenyl-N-(pyridin-4-yl)quinazolin-4-amine (Compound 700); and 2-Phenyl-N-(1H-pyrazol-4-yl)quinazolin-4-amine (Compound 894); or a pharmaceutically acceptable salt or solvate thereof.

In further embodiments, CK1 inhibitors include the imidazoles according to general formula (I) in U.S. Patent Application Publication No. 2005/0203155, including compounds No. 1 to No. 116 recited below, and specifically 4-[4-(4-fluoro-phenyl)-5-pyridine-4-yl-1H-imidazole-2-yl]-phenol, 4-[5-(3-iodo-phenyl)-2-(4-methanesulfinyl-phenyl)-3H-imidazole-4-yl]-pyridine, and 4-[4-(4-fluoro-phenyl)-5-pyridine-4-yl-1H-imidazole-2-yl]-benzylamine.

Compounds No. 1 to No. 116 comprise (Compound 1) 4-[5-(4-Fluoro-phenyl)-2-(4-isopropyl-phenyl)-3H-imidazole-4-yl]-pyridine; (Compound 2) 3-[4-(4-Fluoro-phenyl)-5-pyridine-4-yl-1H-imidazole-2-yl]-4-nitro-phenol; (Compound 3) 4-[4-(4-Fluoro-phenyl)-5-pyridine-4-yl-1H-imidazole-2-yl]-2-nitro-phenol; (Compound 4) 4-[5-(4-Fluoro-phenyl)-2-(3-trifluoromethyl-phenyl)-3H-imidazole-4-yl]-pyridine; (Compound 5) 2,6-Di-tert-butyl-4-[4-(4-fluoro-phenyl)-5-pyridine-4-yl)-1H-imidazole-2-yl]-phenol; (Compound 6) 4-[2-(2,5-Bis-trifluoromethyl)-5-(4-fluoro-phenyl)-3H-imidazole-4-yl]-pyridine; (Compound 7) 4-[5-(4-Fluoro-phenyl)-2-furan-2-yl-3H-imidazole-4-yl]-pyridine; (Compound 8) 4-[5-(4-Fluoro-phenyl)-2-(2-methoxy-phenyl)-3H-imidazole-4-yl]-pyridine; (Compound 9) 4-[5-(4-Fluoro-phenyl)-2-(5-methyl-furan-2-yl)-3H-imidazole-4-yl]-pyridine; (Compound 10) 4-[5-(4-Fluoro-phenyl)-2-(3-methoxy-phenyl)-3H-imidazole-4-yl]-pyridine; (Compound 11) 4-[5-(4-Fluoro-phenyl)-2-p-tolyl-3H-imidazole-4-yl]-pyridine; (Compound 12) 4-[5-(4-Fluoro-phenyl)-2-(4-methoxy-phenyl)-3H-imidazole-4-yl]-pyridine; (Compound 13) 4-[5-(4-Fluoro-phenyl)-2-(2-chloro-phenyl)-3H-imidazole-4-yl]-pyridine; (Compound 14) 4-[5-(4-Fluoro-phenyl)-2-(2,4,6-trimethyl-phenyl)-3H-imidazole-4-yl]-pyridine; (Compound 15) 4-[5-(4-Fluoro-phenyl)-2-(2,4-dichloro-phenyl)-3H-imidazole-4-yl]-pyridine; (Compound 16) 4-[5-(4-Fluoro-phenyl)-2-(2,3-dichloro-phenyl)-3H-imidazole-4-yl]-pyridine; (Compound 17) 4-[4-(4-Fluoro-phenyl)-5-pyridine-4-yl)-1H-imidazole-2-yl]-2-methoxy-phenol; (Compound 18) 4-[5-(4-Fluoro-phenyl)-2-(2-nitro-phenyl)-3H-imidazole-4-yl]-pyridine; (Compound 19) 4-[4-(4-Fluoro-phenyl)-5-pyridine-4-yl-1H-imidazole-2-yl]-benzene-1,2-diol; (Compound 20) 4-[4-(4-Fluoro-phenyl)-5-pyridine-4-yl-1H-imidazole-2-yl]-phenol; (Compound 21) 4-[2-(4,5-Dimethoxy-2-nitro-phenyl)-5-(4-fluoro-phenyl)-3H-imidazole-4-yl]-pyridine; (Compound 22) 4-[5-(4-Fluoro-phenyl)-2-(3-chloro-phenyl)-3H-imidazole-4-yl]-pyridine; (Compound 23) 4-[5-(4-Fluoro-phenyl)-2-(3-bromo-phenyl)-3H-imidazole-4-yl]-pyridine; (Compound 24) 4-[5-(4-Fluoro-phenyl)-2-(3-nitro-phenyl)-3H-imidazole-4-yl]-pyridine; (Compound 25) 4-[5-(4-Fluoro-phenyl)-2-(4-nitro-phenyl)-3H-imidazole-4-yl]-pyridine; (Compound 26) 4-[5-(4-Fluoro-phenyl)-2-naphtalene-1-yl-3H-imidazole-4-yl]-pyridine; (Compound 27) 4-[2-(3,5-Bis-trifluoromethyl-phenyl)-5-(4-fluoro-phenyl)-3H-imidazole-4-yl]-pyridine; (Compound 28) {4-[4-(4-Fluoro-phenyl)-5-pyridine-4-yl-1H-imidazole-2-yl]-phenyl}-dimethyl-amine; (Compound 29) 4-[5-(4-Fluoro-phenyl)-2-(3,4-dichloro-phenyl)-3H-imidazole-4-yl]-pyridine; (Compound 30) 4-[5-(4-Fluoro-phenyl)-2-(4-trifluoromethyl-phenyl)-3H-imidazole-4-yl]-pyridine; (Compound 31) 4-[4-(4-Fluoro-phenyl)-5-pyridine-4-yl-1H-imidazole-2-yl]-2,6-dimethyl-phenol; (Compound 32) 4-[5-(4-Fluoro-phenyl)-2-(4-methyl sulfanyl-phenyl)-3H-imidazole-4-yl]-pyridine; (Compound 33) 3-[4-(4-Fluoro-phenyl)-5-pyridine-4-yl-1H-imidazole-2-yl]-1H-indole; (Compound 34) 4-[5-(4-Fluoro-phenyl)-2-(4-chloro-phenyl)-3H-imidazole-4-yl]-pyridine; (Compound 35) 4-[5-(4-Fluoro-phenyl)-2-thiophene-2-yl-3H-imidazole-4-yl]-pyridine; (Compound 36) 4-[5-(4-Fluoro-phenyl)-2-(4-bromo-phenyl)-3H-imidazole-4-yl]-pyridine; (Compound 37) 4-[2-(3,4-Dimethoxy-phenyl)-5-(4-fluoro-phenyl)-3H-imidazole-4-yl]-pyridine; (Compound 38) 4-[5-(4-Fluoro-phenyl)-2-(4-methanesulfinyl-phenyl)-3H-imidazole-4-yl]-pyridine; (Compound 39) 4-[5-(3-Iodo-phenyl)-2-(4-methanesulfinyl-phenyl)-3H-imidazole-4-yl]-pyridine; (Compound 40) 6-(4-Fluoro-phenyl)-5-pyridine-4-yl-3,7-dihydro-2H-imidazole-[2,1-b]thiazole; (Compound 41) 4-[5-Ethyl-2-(4-methoxy-phenyl)-1H-imidazole-4-yl]-pyridine; (Compound 42) 4-[2,5-Bis-(4-chloro-phenyl)-1H-imidazole-4-yl]-pyridine; (Compound 43) 4-[2-(4-Bromo-phenyl)-5-(4-chloro-phenyl)-1H-imidazole-4-yl]-pyridine; (Compound 44) 4-[2-(2-Chloro-phenyl)-5-(4-chloro-phenyl)-1H-imidazole-4-yl]-pyridine; (Compound 45) 4-[2-(3-Bromo-phenyl)-5-(4-chloro-phenyl)-1H-imidazole-4-yl]-pyridine; (Compound 46) 4-[5-(4-Chloro-phenyl)-2-(2,3-dichloro-phenyl)-1H-imidazole-4-yl]-pyridine; (Compound 47) 3-[5-(4-Chloro-phenyl)-4-pyridine-4-yl-1H-imidazole-2-yl]-4-nitro-phenol; (Compound 48) 4-[5-(4-Chloro-phenyl)-2-(4-fluoro-phenyl)-1H-imidazole-4-yl]-pyridine; (Compound 49) 4-[5-(4-Chloro-phenyl)-2-naphtalene-1-yl-1H-imidazole-4-yl]-pyridine; (Compound 50) 4-[2-(3-Chloro-phenyl)-5-(4-chloro-phenyl)-1H-imidazole-4-yl]-pyridine; (Compound 51) 4-[5-(4-Chloro-phenyl)-2-(3-methoxy-phenyl)-1H-imidazole-4-yl]-pyridine; (Compound 52) 4-[5-(4-Chloro-phenyl)-2-(2-methoxy-phenyl)-1H-imidazole-4-yl]-pyridine; (Compound 53) 4-[5-(4-Chloro-phenyl)-4-pyridine-4-yl-1H-imidazole-2-yl]-benzene-1,3-diol; (Compound 54) 4-[5-(4-Chloro-phenyl)-4-pyridine-4-yl-1H-imidazole-2-yl]-2-methoxy-phenol; (Compound 55) 4-[2-(3-Bromo-phenyl)-5-(3-trifluoromethyl-phenyl)-1H-imidazole-4-yl]-pyridine; (Compound 56) 4-[2-(4-Trifluoromethyl-phenyl)-5-(3-trifluoromethyl-phenyl)-1H-imidazole-4-yl]-pyridine; (Compound 57) 4-[2-(4-Bromo-phenyl)-5-(3-trifluoromethyl-phenyl)-1H-imidazole-4-yl]-pyridine; (Compound 58) 4-[5-(3-Iodo-phenyl)-2-(4-trifluoromethyl-phenyl)-1H-imidazole-4-yl]-pyridine; (Compound 59) 4-[5-(4-Chloro-phenyl)-2-(4-isopropyl-phenyl)-1H-imidazole-4-yl]-pyridine; (Compound 60) 4-[5-(4-Chloro-phenyl)-4-pyridine-4-yl-1H-imidazole-2-yl]-2,6-dimethyl-phenol; (Compound 61) 4-[5-(4-Chloro-phenyl)-2-(2,4-Dichloro phenyl)-1H-imidazole-4-yl]-pyridine; (Compound 62) 4-[5-(4-Chloro-phenyl)-4-pyridine-4-yl-1H-imidazole-2-yl]-benzonitrile; (Compound 63) 4-[5-(4-Chloro-phenyl)-4-pyridine-4-yl-1H-imidazole-2-yl]-phenol; (Compound 64) 2,6-Di-tert-butyl-4-[5-(4-chloro-phenyl)-4-pyridine-4-yl-1H-imidazole-2-yl]-phenol; (Compound 65) 4-[5-(4-Chloro-phenyl)-2-(3,4-dimethoxy-phenyl)-1H-imidazole-4-yl]-pyridine; (Compound 66) 4-[5-(4-Chloro-phenyl)-2-(3-nitro-phenyl)-1H-imidazole-4-yl]-pyridine; (Compound 67) 4-[5-(4-Chloro-phenyl)-2-(3,4-Dichloro phenyl)-1H-imidazole-4-yl]-pyridine; (Compound 68) 4-[5-(4-Chloro-phenyl)-2-(4-methoxy-phenyl)-1H-imidazole-4-yl]-pyridine; (Compound 69) 4-[5-(4-Chloro-phenyl)-4-pyridine-4-yl-1H-imidazole-2-yl]-2,6-diisopropyl-phenol; (Compound 70)N-{4-[5-(4-Chloro-phenyl)-4-pyridine-4-yl-1H-imidazole-2-yl]-acetamide; (Compound 71) 4-[2-(3,4-Dichloro-phenyl)-5-(3-trifluoromethyl-phenyl)-1H-imidazole-4-yl]-pyridine; (Compound 72) 4-[2-(4-Chloro-phenyl)-5-(3-trifluoromethyl-phenyl)-1H-imidazole-4-yl]-pyridine; (Compound 73) 4-[4-Pyridine-4-yl-5-(3-trifluoromethyl-phenyl)-1H-imidazole-2-yl]-phenol; (Compound 74) 4-[4-Pyridine-4-yl-5-(3-trifluoromethyl-phenyl)-1H-imidazole-2-yl]-2-methoxy-phenol; (Compound 75) 4-[2-(3-Chloro-phenyl)-5-(3-trifluoromethyl-phenyl)-1H-imidazole-4-yl]-pyridine; (Compound 76) 4-[2-(4-Methylsulfanyl-phenyl)-5-(3-trifluoromethyl-phenyl)-1H-imidazole-4-yl]-pyridine; (Compound 77) 3-[4-Pyridine-4-yl-5-(3-trifluoromethyl-phenyl)-1H-imidazole-2-yl]-phenol; (Compound 78) 4-[2-(3-Bromo-phenyl)-5-(3-iodo-phenyl)-1H-imidazole-4-yl]-pyridine; (Compound 79) 4-[5-(3-Iodo-phenyl)-4-pyridin-4-yl-1H-imidazole-2-yl]-2,6-dimethyl-phenol; (Compound 80) 4-[2-(4-Bromo-phenyl)-5-(3-iodo-phenyl)-1H-imidazole-4-yl]-pyridine, (Compound 81) 4-[2-(3-Chloro-phenyl)-5-(3-iodo-phenyl)-1H-imidazole-4-yl]-pyridine; (Compound 82) 4-[2-(4-Fluoro-phenyl)-5-(3-iodo-phenyl)-1H-imidazole-4-yl]-pyridine; (Compound 83) 4-[2-Naphtalene-1-yl-5-phenyl-1H-imidazole-4-yl]-pyridine; (Compound 84) 4-(5-Phenyl-2-styryl-1H-imidazole-4-yl]-pyridine; (Compound 85) 4-[5-Phenyl-2-(4-trifluoromethyl-phenyl)-1H-imidazole-4-yl]-pyridine; (Compound 86) 2-Nitro-4-(5-phenyl-4-pyridine-4-yl-1H-imidazole-2-yl)-phenol; (Compound 87) 4-[2-(3-Bromo-phenyl)-5-phenyl-1H-imidazole-4-yl]-pyridine; (Compound 88) 2,6-Dimethyl(5-phenyl-4-pyridine-4-yl-1H-imidazole-2-yl)-phenol; (Compound 89) 4-[2-(3,4-Bis-benzyloxy-phenyl)-5-phenyl-1H-imidazole-4-yl]-pyridine; (Compound 90) 4-[2-(3,4-Dimethoxy-phenyl)-5-phenyl-1H-imidazole-4-yl]-pyridine; (Compound 91) 4-[2-(3-Nitro-phenyl)-5-phenyl-1H-imidazole-4-yl]-pyridine; (Compound 92) 4-[2-(4-Chloro-phenyl)-5-phenyl-1H-imidazole-4-yl]-pyridine; (Compound 93) 2-(5-Phenyl-4-pyridine-4-yl-1H-imidazole-2-yl)-benzene-1,4-diol; (Compound 94) 4-(5-Phenyl-4-pyridine-4-yl-1H-imidazole-2-yl)-phenol; (Compound 95) 3-(5-Phenyl-4-pyridine-4-yl-1H-imidazole-2-yl)-phenol; (Compound 96) 4-[2-(4-Bromo-phenyl)-5-phenyl-1H-imidazole-4-yl]-pyridine; (Compound 97) 2-Methoxy-4-(5-phenyl-4-pyridine-4-yl-1H-imidazole-2-yl)-phenol; (Compound 98) 4-[2-(4-Isopropyl-phenyl)-5-phenyl-1H-imidazole-4-yl]-pyridine; (Compound 99) 4-[2-(2,3-Dichloro-phenyl)-5-phenyl-1H-imidazole-4-yl]-pyridine; (Compound 100) 4-[2-(2,4-Dichloro-phenyl)-5-phenyl-1H-imidazole-4-yl]-pyridine; (Compound 101) 4-[2-(4-Methylsulfanyl-phenyl)-5-phenyl-1H-imidazole-4-yl]-pyridine; (Compound 102) 4-[2-(2-Chloro-phenyl)-5-phenyl-1H-imidazole-4-yl]-pyridine; (Compound 103) 4-[2-(4-Methoxy-phenyl)-5-phenyl-1H-imidazole-4-yl]-pyridine; (Compound 104) 4-[2-(3-Methoxy-phenyl)-5-phenyl-1H-imidazole-4-yl]-pyridine; (Compound 105) 4-[2-(2-Methoxy-phenyl)-5-phenyl-1H-imidazole-4-yl]-pyridine; (Compound 106) 4-[2-(3-Chloro-phenyl)-5-phenyl-1H-imidazole-4-yl]-pyridine; (Compound 107) 2,6-Di-tert-butyl-4-(5-phenyl-4-pyridine-4-yl-1H-imidazole-2-yl)phenol; (Compound 108) 4-(5-Phenyl-4-pyridine-4-yl-1H-imidazole-2-yl)-benzonitrile; (Compound 109)N-[4-(5-Phenyl-4-pyridine-4-yl-1H-imidazole-2-yl)-phenyl]-acetamide; (Compound 110) 4-{2-[2-(2-Methoxy-phenyl)-vinyl]-5-phenyl-1H-imidazole-4-yl}-pyridine; (Compound 111) 4-[5-(3-Iodo-phenyl)4-pyridine-4-yl-1H-imidazole-2-yl]-phenol; (Compound 112) 4-[2-(2,3-Dichloro-phenyl)-5-(3-iodo-phenyl)-1H-imidazole-4-yl]-pyridine; (Compound 113) 4-[5-(4-Chloro-phenyl)-2-(4-methylsulfanyl-phenyl)-1H-imidazole-4-yl]-pyridine; (Compound 114) 4-[5-(4-Chloro-phenyl)4-pyridine-4-yl-1H-imidazole-2-yl]-dimethyl amine; (Compound 115) 4-[5-(3-Iodo-phenyl)-2-(5-methyl-furan-2-yl)-1H-imidazole-4-yl]-pyridine; and (Compound 116) 4-[4-(4-Fluoro-phenyl)-5-pyridine-4-yl-1H-imidazole-2-yl]-benzylamine;

Also useful are compounds No. 117 to No. 173: (Compound 117) 4-[5-(3-Iodo-phenyl)-2-(4-methyl sulfanylphenyl)-1H-imidazole-4-yl]-pyridine; (Compound 118) 4-[2-(4-Methanesulfinyl-phenyl)-5-phenyl-3H-imidazole-4-yl]-pyridine; (Compound 119) 4-[5-(4-Fluoro-phenyl)-4-pyridin-4-yl-3H-imidazole-2-yl]-phenylamine; (Compound 120) {4-[5-(3-Iodo-phenyl)4-pyridin-4-yl-1H-imidazole-2-yl]-phenyl}-methanol; (Compound 121) 4-[5-(4-Fluoro-phenyl)-4-pyridin-4-yl-1H-imidazole-2-yl]-benzylamine; (Compound 122) 2-(3,4-Dimethoxyphenyl)-4,5-bis-(4-methoxyphenyl)-1H-imidazole; (Compound 123) 4-[4,5-Bis-(4-methoxyphenyl)-1H-imidazole-2-yl]-2,6-bis-tert-butylphenol; (Compound 124) 4-[4,5-Bis-(4-methoxyphenyl)-1H-imidazole-2-yl]-2-methoxy-phenol; (Compound 125) 4-[4,5-Bis-(4-bromophenyl)-1H-imidazole-2-yl]-2-methoxy-phenol; (Compound 126) 4-[4,5-Bis-(4-methoxyphenyl)-2-styryl-1H-imidazole; (Compound 127) 4-[4,5-Bis-(4-methoxyphenyl)-2-(4-trifluoromethyl-phenyl)-1H-imidazole]; (Compound 128) 4-[4,5-Bis-(4-methoxyphenyl)-2-(3-trifluoromethyl-phenyl)-1H-imidazole; (Compound 129) 4-[4,5-Bis-(4-methoxyphenyl)-1H-imidazole-2-yl]-2-nitro-phenol; (Compound 130) 4-[4,5-Bis-(4-methoxyphenyl)-1H-imidazole-2-yl]-4-nitro-phenol; (Compound 131) 4-[4,5-Bis-(4-methoxyphenyl)-1H-imidazole-2-yl]-phenol; (Compound 132) 2-(3-Bromo-phenyl)-4,5-bis-(4-methoxyphenyl)-1H-imidazole; (Compound 132) 2-(3,4-Diphenoxy-phenyl)-4,5-bis-(4-methoxyphenyl)-1H-imidazole; (Compound 133) {4-[4,5-Bis-(4-methoxyphenyl)-1H-imidazole-2-yl]-phenyl}-dimethylamine; (Compound 134) 2-(4-Chloro-phenyl)-4,5-bis-(4-methoxyphenyl)-1H-imidazole; (Compound 135) 2-(4-Bromo-phenyl)-4,5-bis-(4-methoxyphenyl)-1H-imidazole; (Compound 136) 4,5-Bis-(4-methoxyphenyl)-2-(3-nitrophenyl)-1H-imidazole; (Compound 137) 4,5-Bis-(4-methoxyphenyl)-2-naphthalen-1-yl-1H-imidazole; (Compound 138) 2-(2,3-Dichlorophenyl)-4,5-bis-(4-methoxyphenyl)-1H-imidazole; (Compound 139) 2-(2,4-Dichlorophenyl)-4,5-bis-(4-methoxyphenyl)-1H-imidazole; (Compound 140) 4,5-Bis-(4-methoxyphenyl)-2-(4-nitro-phenyl)-H-imidazole; (Compound 141) 4-[4,5-Bis-(4-methoxyphenyl)-1H-imidazole-2-yl]-benzene-1,2-diol; (Compound 142) 2-(4-Methoxy-3,5-dimethyl-phenyl)-4,5-bis-(4-methoxy-phenyl)-1H-imidazole; (Compound 143) 4-[4,5-Bis-(4-methoxyphenyl)-1H-imidazole-2-yl]-1H-indole; (Compound 144) 2-(3,4-Bis-benzyloxy-phenyl)-4,5-bis-(4-bromo-phenyl)-1H-imidazole; (Compound 145) 4,5-Bis-(4-bromo-phenyl)-2-(4-isopropyl-phenyl)-1H-imidazole; (Compound 146) 4,5-Bis-(4-bromo-phenyl)-2-(2,4-dichloro-phenyl)-1H-imidazole; (Compound 147) 4,5-Bis-(4-bromo-phenyl)-2-(4-chloro-phenyl)-1H-imidazole; (Compound 148) 4,5-Bis-(4-bromo-phenyl)-2-(4-trifluoromethyl-phenyl)-1H-imidazole; (Compound 149) 4,5-Bis-(4-bromo-phenyl)-2-(3-trifluoromethyl-phenyl)-1H-imidazole; (Compound 150) 2-(3,5-Bis-trifluoromethyl-phenyl)-4,5-bis-(4-bromo-phenyl)-1H-imidazole; (Compound 151) 2-(3,5-Bis-trifluoromethyl-phenyl)-4,5-bis-(4-bromo-phenyl)-1H-imidazole; (Compound 152) 4,5-Bis-(4-bromo-phenyl)-2-(3,4-dimethoxy-phenyl)-1H-imidazole; (Compound 153) 4,5-Bis-(4-bromo-phenyl)-2-(4-methyl sulfanyl-phenyl)-1H-imidazole; (Compound 154) 2-(3-Bromo-phenyl)-4,5-bis-(4-bromo-phenyl)-1H-imidazole; (Compound 155) 4,5-Bis-(4-bromo-phenyl)-2-(2,3-dichloro-phenyl)-1H-imidazole; (Compound 156) 4,5-Bis-(4-bromo-phenyl)-2-(3-nitro-phenyl)-1H-imidazole; (Compound 157) 4-[4,5-Bis-(4-bromo-phenyl)-1H-imidazole-2-yl]-2,6-dimethyl-phenol; (Compound 158) 4,5-Bis-(4-bromo-phenyl)-2-(4,5-dimethoxy-2-nitro-phenyl)-1H-imidazole; (Compound 159) 4-[4,5-Bis-(4-bromo-phenyl)-1H-imidazole-2-yl]-2-nitro-phenol; (Compound 160) {4-[4,5-Bis-(4-bromo-phenyl)-1H-imidazole-2-yl]-phenyl}-dimethylamine; (Compound 161) 4,5-Bis-(4-bromo-phenyl)-2-naphthalen-1-yl-1H-imidazole; (Compound 162) 4,5-Bis-(4-bromo-phenyl)-2-(5-ethyl-furan-2-yl)-1H-imidazole; (Compound 163) 4,5-Bis-(4-bromo-phenyl)-2-thiophen-2-yl-1H-imidazole; (Compound 164) 3-[4,5-Bis-(4-bromophenyl)-1H-imidazole-2-yl]-1H-indole; (Compound 165) 2-(3,4-Dimethoxy-phenyl)-4,5-di-thiophen-2-yl-1H-imidazole; (Compound 166) 2-(4-Isopropyl-phenyl)-4,5-di-thiophen-2-yl-1H-imidazole; (Compound 167) 2-(3-Bromo-phenyl)-4,5-di-thiophen-2-yl-1H-imidazole; (Compound 168) 4,5-Di-thiophen-2-yl-2-(4-trifluoromethyl-phenyl)-1H-imidazole; (Compound 169) 4,5-Di-thiophen-2-yl-2-(3-trifluoromethyl-phenyl)-1H-imidazole; (Compound 170) [4-(4,5-Di-thiophen-2-yl-1H-imidazole-2-yl)-phenyl]-dimethylamine; (Compound 171) 2-(3,4-Bis-benzyloxy-phenyl)-4,5-di-thiophen-2-yl-1H-imidazole; (Compound 172) 2-Naphthalen-1-yl-4,5-di-thiophen-2-yl-1H-imidazole; and (Compound 173) 4-(4,5-Di-thiophen-2-yl-1H-imidazole-2-yl)-2-nitrophenol.

Other CK1 inhibitors useful in the methods of the present invention include those described in U.S. Pat. No. 9,233,965 (Sanofi) including, but not limited to, N²-tert-butyl-7-phenyl-3,4-dihydropyrrolo[1,2-a]pyrazine-2,8(1H)-dicarboxamide; N²-tert-butyl-7-(4-methoxyphenyl)-3,4-dihydropyrrolo[1,2-a]pyrazine-2,8(1H)-dicarboxamide; N²-tert-butyl-6-methyl-7-phenyl-3,4-dihydropyrrolo[1,2-a]pyrazine-2,8(1H)-dicarboxamide; N²-tert-butyl-7-(4-methoxyphenyl)-6-methyl-3,4-dihydropyrrolo[1,2-a]pyrazine-2,8(1H)-dicarboxamide; N²-tert-butyl-6-methyl-7-(4-phenoxyphenyl)-3,4-dihydropyrrolo[1,2-a]pyrazine-2,8(1H)-dicarboxamide; N²-6-di-cyclo-propyl-7-(4-methoxyphenyl)-3,4-dihydropyrrolo[1,2-a]pyrazine-2,8(1H)-dicarboxamide; 6-cyclo-propyl-7-phenyl-N²-(iso-propyl)-3,4-dihydropyrrolo[1,2-a]pyrazine-2,8(1H)-dicarboxamide; 6-cyclo-propyl-7-(4-methoxyphenyl)-N²-(iso-propyl)-3,4-dihydropyrrolo[1,2-a]pyrazine-2,8(1H)-dicarboxamide; 6-cyclo-propyl-7-phenyl-N²-(iso-butyl)-3,4-dihydropyrrolo[1,2-a]pyrazine-2,8(1H)-dicarboxamide; N²-tert-butyl-6-cyclo-propyl-7-phenyl-3,4-dihydropyrrolo[1,2-a]pyrazine-2,8(1H)-dicarboxamide; N²-tert-butyl-6-cyclo-propyl-7-(3-methylphenyl)-3,4-dihydropyrrolo[1,2-a]pyrazine-2,8(1H)-dicarboxamide; N²-tert-butyl-6-cyclo-propyl-7-(4-methylphenyl)-3,4-dihydropyrrolo[1,2-a]pyrazine-2,8(1H)-dicarboxamide; N²-tert-butyl-6-cyclo-propyl-7-[4-(iso-propyl)phenyl]-3,4-dihydropyrrolo[1,2-a]pyrazine-2,8(1H)-dicarboxamide; N²-tert-butyl-7-(4-cyclo-hexylphenyl)-6-cyclo-propyl-3,4-dihydropyrrolo[1,2-a]pyrazine-2,8(1H)-dicarboxamide; 7-(biphenyl-4-yl)-N²-tert-butyl-6-cyclo-propyl-3,4-dihydropyrrolo[1,2-a]pyrazine-2,8(1H)-dicarboxamide; N²-tert-butyl-6-cyclo-propyl-7-[3-(trifluoromethyl)phenyl]-3,4-dihydropyrrolo[1,2-a]pyrazine-2,8(1H)-dicarboxamide; N²-tert-butyl-6-cyclo-propyl-7-[3-(dimethylcarbamoyl)phenyl]-3,4-dihydropyrrlo[1,2-a]pyrazine-2,8(1H)-dicarboxamide; N²-tert-butyl-6-cyclo-propyl-7-[4-(cyclo-propylcarbamoyl)phenyl]-3,4-dihydropyrrolo[1,2-a]pyrazine-2,8(1H)-dicarboxamide; N²-tert-butyl-6-cyclo-propyl-7-[4-(pyrrolidin-1-ylcarbonyl)phenyl]-3,4-dihydropyrrolo[1,2-a]pyrazine-2,8(1H)-dicarboxamide; N²-tert-butyl-6-cyclo-propyl-7-{4-[methoxy(methyl)carbamoyl]phenyl}-3,4-dihydropyrrolo[1,2-a]pyrazine-2,8(1H)-dicarboxamide; N²-tert-butyl-7-(3-cyanophenyl)-6-cyclo-propyl-3,4-dihydropyrrolo[1,2-a]pyrazine-2,8(1H)-dicarboxamide; N²-tert-butyl-7-(4-cyanophenyl)-6-cyclo-propyl-3,4-dihydropyrrolo[1,2-a]pyrazine-2,8(1H)-dicarboxamide; N²-tert-butyl-6-cyclo-propyl-7-[4-(5-methyl-1,3,4-oxadiazol-2-yl)phenyl]-3,4-dihydropyrrolo[1,2-a]pyrazine-2,8(1H)-dicarboxamide; N²-tert-butyl-6-cyclo-propyl-7-[4-(trifluoromethyl)phenyl]-3,4-dihydropyrrolo[1,2-a]pyrazine-2,8(1H)-dicarboxamide; N²-tert-butyl-6-cyclo-propyl-7-(naphthalen-2-yl)-3,4-dihydropyrrolo[1,2-a]pyrazine-2,8(1H)-dicarboxamide; N²-tert-butyl-6-cyclo-propyl-7-{3-[(dimethylsulfamoyl)amino]phenyl}-3,4-dihydropyrrolo[1,2-a]pyrazine-2,8(1H)-dicarboxamide; N²-tert-butyl-6-cyclo-propyl-7-[3-(1H-pyrazol-1-yl)phenyl]-3,4-dihydropyrrolo[1,2-a]pyrazine-2,8(1H)-dicarboxamide; N²-tert-butyl-6-cyclo-propyl-7-[4-(dimethylamino)phenyl]-3,4-dihydropyrrolo[1,2-a]pyrazine-2,8(1H)-dicarboxamide; N²-tert-butyl-6-cyclo-propyl-7-{4-[(methylsulfonyl)amino]phenyl}-3,4-dihydropyrrolo[1,2-a]pyrazine-2,8(1H)-dicarboxamide; N²-tert-butyl-6-cyclo-propyl-7-[4-(morpholin-4-yl)phenyl]-3,4-dihydropyrrolo[1,2-a]pyrazine-2,8(1H)-dicarboxamide; N²-tert-butyl-6-cyclo-propyl-7-[4-(1H-pyrazol-1-yl)phenyl]-3,4-dihydropyrrolo[1,2-a]pyrazine-2,8(1H)-dicarboxamide; N²-tert-butyl-6-cyclo-propyl-7-(3-methoxyphenyl)-3,4-dihydropyrrolo[1,2-a]pyrazine-2,8(1H)-dicarboxamide; N²-tert-butyl-6-cyclo-propyl-7-[3-(cyclo-propylmethoxy)phenyl]-3,4-dihydropyrrolo[1,2-a]pyrazine-2,8(1H)-dicarboxamide; 7-[3-(benzyloxy)phenyl]-N²-tert-butyl-6-cyclo-propyl-3,4-dihydropyrrolo[1,2-a]pyrazine-2,8(1H)-dicarboxamide; N²-tert-butyl-6-cyclo-propyl-7-[3-(trifluoromethoxy)phenyl]-3,4-dihydropyrrolo[1,2-a]pyrazine-2,8(1H)-dicarboxamide; N²-tert-butyl-6-cyclo-propyl-7-[4-(methoxy)phenyl]-3,4-dihydropyrrolo[1,2-a]pyrazine-2,8(1H)-dicarboxamide; N²-tert-butyl-6-cyclo-propyl-7-[4-(cyclo-propylmethoxy)phenyl]-3,4-dihydropyrrolo[1,2-a]pyrazine-2,8(1H)-dicarboxamide; 7-(4-butoxyphenyl)-N²-tert-butyl-6-cyclo-propyl-3,4-dihydropyrrolo[1,2-a]pyrazine-2,8(1H)-dicarboxamide; N²-tert-butyl-6-cyclo-propyl-7-(4-phenoxyphenyl)-3,4-dihydropyrrolo[1,2-a]pyrazine-2,8(1H)-dicarboxamide; 7-[4-(benzyloxy)phenyl]-N²-tert-butyl-6-cyclo-propyl-3,4-dihydropyrrlo[1,2-a]pyrazine-2,8(1H)-dicarboxamide; N²-tert-butyl-6-cyclo-propyl-7-{4-[(4-fluorobenzyl)oxy]phenyl}-3,4-dihydropyrrolo[1,2-a]pyrazine-2,8(1H)-dicarboxamide; N²-tert-butyl-7-{3-chloro-4-[(4-fluorobenzyl)oxy]phenyl}-6-cyclo-propyl-3,4-dihydropyrrolo[1,2-a]pyrazine-2,8(1H)-dicarboxamide; N²-tert-butyl-7-[4-(cyanomethoxy)phenyl]-6-cyclo-propyl-3,4-dihydropyrrolo[1,2-a]pyrazine-2,8(1H)-dicarboxamide; N²-tert-butyl-6-cyclo-propyl-7-[4-(trifluoromethoxy)phenyl]-3,4-dihydropyrrolo[1,2-a]pyrazine-2,8(1H)-dicarboxamide; N²-tert-butyl-6-cyclo-propyl-7-(2-fluorophenyl)-3,4-dihydropyrrolo[1,2-a]pyrazine-2,8(1H)-dicarboxamide; N²-tert-butyl-6-cyclo-propyl-7-(3-fluorophenyl)-3,4-dihydropyrrolo[1,2-a]pyrazine-2,8(1H)-dicarboxamide; N²-tert-butyl-6-cyclo-propyl-7-(4-fluorophenyl)-3,4-dihydropyrrolo[1,2-a]pyrazine-2,8(1H)-dicarboxamide; N²-tert-butyl-6-cyclo-propyl-7-[4-(methylsulfanyl)phenyl]-3,4-dihydropyrrolo[1,2-a]pyrazine-2,8(1H)-dicarboxamide; N²-cyclo-hexyl-6-cyclo-propyl-7-phenyl-3,4-dihydropyrrolo[1,2-a]pyrazine-2,8(1H)-dicarboxamide; N²-(cyclo-hexylmethyl)-6-cyclo-propyl-7-phenyl-3,4-dihydropyrrolo[1,2-a]pyrazine-2,8(1H)-dicarboxamide; N²-[1,1-bi(cyclo-propyl)-1-yl]-6-cyclo-propyl-7-(4-methoxyphenyl)-3,4-dihydropyrrolo[1,2-a]pyrazine-2,8(1H)-dicarboxamide; 6-cyclo-propyl-7-phenyl-N²-(2,4,4-trimethylpentan-2-yl)-3,4-dihydropyrrolo[1,2-a]pyrazine-2,8(1H)-dicarboxamide; 6-cyclo-propyl-N²-(hexahydro-2,5-methanopentalen-3a(1H)-yl)-7-(4-methoxyphenyl)-3,4-dihydropyrrolo[1,2-a]pyrazine-2,8(1H)-dicarboxamide; N²-(adamantan-1-yl)-6-cyclo-propyl-7-phenyl-3,4-dihydropyrrolo[1,2-a]pyrazine-2,8(1H)-dicarboxamide; N²-(adamantan-1-yl)-6-cyclo-propyl-7-(4-methoxyphenyl)-3,4-dihydropyrrolo[1,2-a]pyrazine-2,8(1H)-dicarboxamide; 6-cyclo-propyl-7-(4-methoxyphenyl)-N²-(tetrahydro-2H-pyran-4-yl)-3,4-dihydropyrrolo[1,2-a]pyrazine-2,8(1H)-dicarboxamide; 6-cyclo-propyl-N²-(1-methoxy-2-methylpropan-2-yl)-7-(4-methoxyphenyl)-3,4-dihydropyrrolo[1,2-a]pyrazine-2,8(1H)-dicarboxamide; N²-tert-butyl-6-fluoro-7-phenyl-3,4-dihydropyrrolo[1,2-a]pyrazine-2,8(1H)-dicarboxamide; 6-chloro-N²-iso-butyl-7-phenyl-3,4-dihydropyrrolo[1,2-a]pyrazine-2,8(1H)-dicarboxamide; N²-tert-butyl-6-chloro-7-phenyl-3,4-dihydropyrrolo[1,2-a]pyrazine-2,8(1H)-dicarboxamide; N²-tert-butyl-6-chloro-7-(3-methylphenyl)-3,4-dihydropyrrolo[1,2-a]pyrazine-2,8(1H)-dicarboxamide; N²-tert-butyl-6-chloro-7-(3-methoxyphenyl)-3,4-dihydropyrrolo[1,2-a]pyrazine-2,8(1H)-dicarboxamide; N²-tert-butyl-6-chloro-7-[3-(trifluoromethoxy)phenyl]-3,4-dihydropyrrolo[1,2-a]pyrazine-2,8(1H)-dicarboxamide; N²-tert-butyl-6-chloro-7-(4-methoxyphenyl)-3,4-dihydropyrrolo[1,2-a]pyrazine-2,8(1H)-dicarboxamide; N²-tert-butyl-6-chloro-7-(3-cyanophenyl)-3,4-dihydropyrrolo[1,2-a]pyrazine-2,8(1H)-dicarboxamide; N²-tert-butyl-6-chloro-7-[3-(trifluoromethyl)phenyl]-3,4-dihydropyrrolo[1,2-a]pyrazine-2,8(1H)-dicarboxamide; N²-tert-butyl-6-chloro-7-(3-fluorophenyl)-3,4-dihydropyrrolo[1,2-a]pyrazine-2,8(1H)-dicarboxamide; 6-chloro-N²-(cyclo-propylmethyl)-7-phenyl-3,4-dihydropyrrolo[1,2-a]pyrazine-2,8(1H)-dicarboxamide; 6-chloro-N²-(3-methylbutyl)-7-phenyl-3,4-dihydropyrrolo[1,2-a]pyrazine-2,8(1H)-dicarboxamide; 6-chloro-N²-(2,2-dimethylpropyl)-7-phenyl-3,4-dihydropyrrolo[1,2-a]pyrazine-2,8(1H)-dicarboxamide; 6-chloro-N²-(2-ethylbutyl)-7-phenyl-3,4-dihydropyrrolo[1,2-a]pyrazine-2,8(1H)-dicarboxamide; 6-chloro-N²-(3,3-dimethylbutyl)-7-phenyl-3,4-dihydropyrrolo[1,2-a]pyrazine-2,8(1H)-dicarboxamide; 6-chloro-7-(3-fluorophenyl)-N²-(3-hydroxy-2,2-dimethylpropyl)-3,4-dihydropyrrolo[1,2-a]pyrazine-2,8(1H)-dicarboxamide; 6-chloro-N²-(3-hydroxy-2,2-dimethylpropyl)-7-(3-trifluoromethyl-phenyl)-3,4-dihydropyrrolo[1,2-a]pyrazine-2,8(1H)-dicarboxamide; 6-chloro-7-(3-fluorophenyl)-N²-{[1-(hydroxymethyl)cyclo-propyl]methyl}-3,4-dihydropyrrolo[1,2-a]pyrazine-2,8(1H)-dicarboxamide; 6-chloro-7-phenyl-N²-(2,2,2-trifluoroethyl)-3,4-dihydropyrrolo[1,2-a]pyrazine-2,8(1H)-dicarboxamide; 6-chloro-7-(3-fluorophenyl)-N²-(2,2,2-trifluoroethyl)-3,4-dihydropyrrolo[1,2-a]pyrazine-2,8(1H)-dicarboxamide; 6-chloro-7-(3-fluorophenyl)-N²-[(2S)-1,1,1-trifluoropropan-2-yl]-3,4-dihydropyrrolo[1,2-a]pyrazine-2,8(1H)-dicarboxamide; 6-chloro-7-(3-fluorophenyl)-N²-(1,1,1-trifluoro-2-methylpropan-2-yl)-3,4-dihydropyrrolo[1,2-a]pyrazine-2,8(1H)-dicarboxamide; 6-chloro-7-(3-fluorophenyl)-N²-(3,3,3-trifluoropropyl)-3,4-dihydropyrrolo[1,2-a]pyrazine-2,8(1H)-dicarboxamide; 6-chloro-7-(3-fluorophenyl)-N²-(4,4,4-trifluorobutyl)-3,4-dihydropyrrolo[1,2-a]pyrazine-2,8(1H)-dicarboxamide; 6-chloro-N²-cyclo-hexyl-7-phenyl-3,4-dihydropyrrolo[1,2-a]pyrazine-2,8(1H)-dicarboxamide; trans-6-chloro-7-(3-fluorophenyl)-N²-(4-hydroxy-cyclo-hexyl)-3,4-dihydropyrrolo[1,2-a]pyrazine-2,8(1H)-dicarboxamide; trans-6-chloro-7-(3-fluorophenyl)-N²-(4-hydroxy-4-methyl-cyclo-hexyl)-3,4-dihydropyrrolo[1,2-a]pyrazine-2,8(1H)-dicarboxamide; trans-6-chloro-7-(3-fluorophenyl)-N²-(4-hydroxy-4-trifluoromethyl-cyclo-hexyl)-3,4-dihydropyrrolo[1,2-a]pyrazine-2,8(1H)-dicarboxamide; trans-6-chloro-7-(3-fluorophenyl)-N²-(4-methoxyimino-cyclohexyl)-3,4-dihydropyrrolo[1,2-a]pyrazine-2,8(1H)-dicarboxamide; trans-6-chloro-7-(3-fluorophenyl)-N²-(4-tert-butyloxyimino-cyclohexyl)-3,4-dihydropyrrolo[1,2-a]pyrazine-2,8(1H)-dicarboxamide; N²-(bicyclo[2.2.1]hept-2-yl)-6-chloro-7-phenyl-3,4-dihydropyrrolo[1,2-a]pyrazine-2,8(1H)-dicarboxamide; 6-chloro-N²-(4,4-difluoro-cyclo-hexyl)-7-(3-fluorophenyl)-3,4-dihydropyrrolo[1,2-a]pyrazine-2,8(1H)-dicarboxamide; 6-chloro-7-(3-fluorophenyl)-N²-(oxetan-3-yl)-3,4-dihydropyrrolo[1,2-a]pyrazine-2,8(1H)-dicarboxamide; 6-chloro-7-(3-fluorophenyl)-N²-(3-methyl-oxetan-3-ylmethyl)-3,4-dihydropyrrolo[1,2-a]pyrazine-2,8(1H)-dicarboxamide; 6-chloro-7-phenyl-N²-(tetrahydrofuran-3-yl)-3,4-dihydropyrrolo[1,2-a]pyrazine-2,8(1H)-dicarboxamide; 6-chloro-7-phenyl-N²-(tetrahydro-2H-pyran-4-yl)-3,4-dihydropyrrolo[1,2-a]pyrazine-2,8(1H)-dicarboxamide; 6-chloro-7-(3-methylphenyl)-N²-(tetrahydro-2H-pyran-4-yl)-3,4-dihydropyrrolo[1,2-a]pyrazine-2,8(1H)-dicarboxamide; 6-chloro-7-(3-cyanophenyl)-N²-(tetrahydro-2H-pyran-4-yl)-3,4-dihydropyrrolo[1,2-a]pyrazine-2,8(1H)-dicarboxamide; 6-chloro-N²-(tetrahydro-2H-pyran-4-yl)-7-[3-(trifluoromethyl)phenyl]-3,4-dihydropyrrolo[1,2-a]pyrazine-2,8(1H)-dicarboxamide; 6-chloro-N²-(tetrahydro-2H-pyran-4-yl)-7-[3-(trifluoromethoxy)phenyl]-3,4-dihydropyrrolo[1,2-a]pyrazine-2,8(1H)-dicarboxamide; 6-chloro-7-(3-fluorophenyl)-N²-(tetrahydro-2H-pyran-4-yl)-3,4-dihydropyrrolo[1,2-a]pyrazine-2,8(1H)-dicarboxamide; 6-chloro-7-(3-fluorophenyl)-N²-(2,2-dimethyl-tetrahydro-2H-pyran-4-yl)-3,4-dihydropyrrolo[1,2-a]pyrazine-2,8(1H)-dicarboxamide; cis-6-chloro-N²-(2,6-dimethyl-tetrahydro-2H-pyran-4-yl)-7-(3-fluorophenyl)-3,4-dihydropyrrolo[1,2-a]pyrazine-2,8(1H)-dicarboxamide; 6-chloro-N²-(1,1-dioxydotetrahydrothiophen-3-yl)-7-phenyl-3,4-dihydropyrrolo[1,2-a]pyrazine-2,8(1H)-dicarboxamide; 6-chloro-N²-(1,1-dioxydotetrahydrothiophen-3-yl)-7-(3-fluorophenyl)-3,4-dihydropyrrolo[1,2-a]pyrazine-2,8(1H)-dicarboxamide; 6-chloro-N²-(1,1-dioxydotetrahydro-2H-thiopyran-4-yl)-7-(3-fluorophenyl)-3,4-dihydropyrrolo[1,2-a]pyrazine-2,8(1H)-dicarboxamide; 6-bromo-7-(3-fluorophenyl)-N²-tert-butyl-3,4-dihydropyrrolo[1,2-a]pyrazine-2,8(1H)-dicarboxamide; 6-cyano-7-(3-fluorophenyl)-N²-(tert-butyl)-3,4-dihydropyrrolo[1,2-a]pyrazine-2,8(1H)-dicarboxamide; 6-cyano-7-(3-fluorophenyl)-N²-((2S)-1,1,1-trifluoropropan-2-yl)-3,4-dihydropyrrolo[1,2-a]pyrazine-2,8(1H)-dicarboxamide; 6-cyano-7-(3-fluorophenyl)-N²-(1,1,1-trifluoro-2-methylpropan-2-yl)-3,4-dihydropyrrolo[1,2-a]pyrazine-2,8(1H)-dicarboxamide; 6-cyano-7-(3-fluorophenyl)-N²-(4,4,4-trifluoro-butyl)-3,4-dihydropyrrolo[1,2-a]pyrazine-2,8(1H)-dicarboxamide; 6-cyano-7-(3-fluorophenyl)-N²-(tetrahydro-2H-pyran-4-yl)-3,4-dihydropyrrolo[1,2-a]pyrazine-2,8(1H)-dicarboxamide; and 6-cyano-N²-(4,4-difluoro-cyclohexyl)-7-(3-fluorophenyl)-3,4-dihydropyrrolo[1,2-a]pyrazine-2,8(1H)-dicarboxamide; in the form of the base or of an addition salt with a pharmaceutically acceptable acid.

MEK/ERK inhibitors include, but are not limited to, BRAF inhibitors (vemurafenib, dabrafenib, LGX818), MEK inhibitors (trametinib, selumetinib, MEK162, PD98059, U0126, CI-1040 (PD184352), PD325901, AZD6244) and MAPK/ERK inhibitors. MAPK/ERK inhibitors include, but are not limited to, R04402257 (Hoffman-LaRoche), R03201195 (Hoffman-LaRoche), PH-797804 (Pfizer), AZD-6703 (AstraZeneca), GW681323 (GlaxoSmithKline), VX-745 (Vertex Pharmaceuticals), VX-702 (Vertex Pharmaceuticals), Scio-469 (Scios/Johnson & Johnson), Scio-323 (Scios/Johnson & Johnson), TAK-715 (Takeda Pharmaceuticals), RWJ-67657 (Johnson & Johnson), BIRB-796 (Boehringer Ingelheim), KC706 (Kemia), ARRY-797 (Array BioPharma), AMG-548 (Amgen), CC-401 (Celgene), SP600125 (Celgene), CEP-1347 (Cephalon), Sorafenib Bayer/Onyx), Raf265 (Novartis), PLX4032 (Roche/Plexxikon), PD0325901 (Pfizer), ARRY-142886 (Array BioPharma), AZD6244 (AstraZeneca/Array Biopharma), ARRY-438162 (Array BioPharma), RDEA119/BAY-869766 (Ardea Biosciences/Bayer), and RDEA436 (Ardea Biosciences).

While the Examples highlight use of the BRD4 inhibitors, CD1 inhibitors and MEK/ERK inhibitors, agents for use in the methods of the invention may encompass those from numerous chemical classes, such as small organic molecules, peptides, saccharides, fatty acids, steroids, purines, pyrimidines and the like. In one aspect, an agent for use in with the present invention is a polynucleotide, such as an antisense oligonucleotide or RNA molecule. In various aspects, the agent may be a polynucleotide, such as an antisense oligonucleotide or RNA molecule, such as microRNA, dsRNA, siRNA, stRNA, and shRNA.

MicroRNAs (miRNA) are single-stranded RNA molecules, which regulate gene expression. miRNAs are encoded by genes from whose DNA they are transcribed but miRNAs are not translated into protein; instead each primary transcript (a pri-miRNA) is processed into a short stem-loop structure called a pre-miRNA and finally into a functional miRNA. Mature miRNA molecules are either fully or partially complementary to one or more messenger RNA (mRNA) molecules, and their main function is to down-regulate gene expression. MicroRNAs can be encoded by independent genes, but also be processed (via the enzyme Dicer) from a variety of different RNA species, including introns, 3′ UTRs of mRNAs, long noncoding RNAs, snoRNAs and transposons. As used herein, microRNAs also include “mimic” microRNAs which are intended to mean a microRNA exogenously introduced into a cell that have the same or substantially the same function as their endogenous counterpart. Thus, while one of skill in the art would understand that an agent may be an exogenously introduced RNA, an agent also includes a compound or the like that increase or decrease expression of microRNA in the cell.

The terms “small interfering RNA” and “siRNA” also are used herein to refer to short interfering RNA or silencing RNA, which are a class of short double-stranded RNA molecules that play a variety of biological roles. Most notably, siRNA is involved in the RNA interference (RNAi) pathway where the siRNA interferes with the expression of a specific gene. In addition to their role in the RNAi pathway, siRNAs also act in RNAi-related pathways (e.g., as an antiviral mechanism or in shaping the chromatin structure of a genome).

Polynucleotides of the present invention, such as antisense oligonucleotides and RNA molecules may be of any suitable length. For example, one of skill in the art would understand what lengths are suitable for antisense oligonucleotides or RNA molecule to be used to regulate gene expression. Such molecules are typically from about 5 to 100, 5 to 50, 5 to 45, 5 to 40, 5 to 35, 5 to 30, 5 to 25, 5 to 20, or 10 to 20 nucleotides in length. For example the molecule may be about 5, 10, 15, 16, 17, 18, 19, 20, 21, 22, 23, 24, 25, 26, 27, 28, 29, 30, 31, 32, 33, 34, 35, 40, 45 or 50 nucleotides in length. Such polynucleotides may include from at least about 15 to more than about 120 nucleotides, including at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides or greater than 120 nucleotides.

The term “polynucleotide” or “nucleotide sequence” or “nucleic acid molecule” is used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. Depending on the use, however, a polynucleotide also can contain nucleotide analogs, including non-naturally occurring synthetic nucleotides or modified naturally occurring nucleotides. Nucleotide analogs are well known in the art and commercially available, as are polynucleotides containing such nucleotide analogs. The covalent bond linking the nucleotides of a polynucleotide generally is a phosphodiester bond. However, depending on the purpose for which the polynucleotide is to be used, the covalent bond also can be any of numerous other bonds, including a thiodiester bond, a phosphorothioate bond, a peptide-like bond or any other bond known to those in the art as useful for linking nucleotides to produce synthetic polynucleotides.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template.

In various embodiments antisense oligonucleotides or RNA molecules include oligonucleotides containing modifications. A variety of modification are known in the art and contemplated for use in the present invention. For example oligonucleotides containing modified backbones or non-natural internucleoside linkages are contemplated. As used herein, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In various aspects modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2-5′ linked analogs of these, and those having inverted polarity wherein one or more internucleotide linkages is a 3′ to 3′,5′ to 5′ or 2′ to 2′ linkage. Certain oligonucleotides having inverted polarity comprise a single 3′ to 3′ linkage at the 3′-most internucleotide linkage i.e., a single inverted nucleoside residue which may be abasic (the nucleobase is missing or has a hydroxyl group in place thereof). Various salts, mixed salts and free acid forms are also included.

In various aspects modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In various aspects, oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. In various aspects, oligonucleotides may include phosphorothioate backbones and oligonucleosides with heteroatom backbones. Modified oligonucleotides may also contain one or more substituted sugar moieties. In some embodiments oligonucleotides comprise one of the following at the 2′ position: OH; F; 0-, S-, or N-alkyl; 0-, S-, or N-alkenyl; 0-, S- or N-alkynyl; or O-alkyl-O-alkyl, wherein the alkyl, alkenyl and alkynyl may be substituted or unsubstituted Ci to C₁₀ alkyl or C₂ to C₁₀ alkenyl and alkynyl. Particularly preferred are 0[(CH₂)ₙO]ₘCH₃, 0(CH₂)ₙOCH₃, 0(CH₂)ₙH₂, 0(CH₂)ₙCH₃, 0(CH₂)ₙOH₂ and 0(CH₂)ₙON[(CH₂)ₙCH₃)]₂, where n and m are from 1 to about 10. Other preferred oligonucleotides comprise one of the following at the 2′ position: Ci to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, S0₂CH₃, ON0₂, N0₂, N3, H₂, heterocycloalkyl, heterocycloalkaryl, aminoalkyl amino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Another modification includes 2′-methoxyethoxy (2′OCH₂CH₂0CH₃, also known as 2′-0-(2-methoxyethyl) or 2′-MOE).

In one embodiment, an agent features a chemically modified nucleic acid molecule that includes one or more chemical modifications described herein. Non-limiting examples of such chemical modifications include without limitation phosphorothioate internucleotide linkages, 2′-deoxyribonucleotides, 2′-0-methyl ribonucleotides, 2′-deoxy-2′-fluoro ribonucleotides, “universal base” nucleotides, “acyclic” nucleotides, 5′-C-methyl nucleotides, and terminal glyceryl and/or inverted deoxy abasic residue incorporation. These chemical modifications are shown to preserve activity in cells while at the same time, dramatically increasing the serum stability of these compounds. In one aspect, the chemically modified nucleotide used in the invention includes a 2′-deoxyribonucleotide, 2′-O-methyl ribonucleotide, 2′-fluoro ribonucleotide, 2′-amino ribonucleotide, 2′-0-amino ribonucleotide, 2′-C-allyl ribonucleotide, 2′-0-allyl ribonucleotide, 2′-methoxyethyl ribonucleotide, 5′-C-methyl ribonucleotide, or a combination thereof. In another aspect, the chemically modified oligonucleotide used in the invention includes a 2′-deoxyribonucleotide, 2′-0-methyl ribonucleotide, 2′-fluoro ribonucleotide, 2′-amino ribonucleotide, 2′-0-amino ribonucleotide, 2′-C-allyl ribonucleotide, 2′-0-allyl ribonucleotide, 2′-methoxyethyl ribonucleotide, 5′-C-methyl ribonucleotide, or a combination thereof.

In a non-limiting example, the introduction of chemically modified nucleotides into nucleic acid molecules provides a powerful tool in overcoming potential limitations of in vivo stability and bioavailability inherent to native RNA molecules that are delivered exogenously. For example, the use of chemically modified nucleic acid molecules can enable a lower dose of a particular nucleic acid molecule for a given therapeutic effect since chemically modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically modified nucleic acid molecule is reduced as compared to a native nucleic acid molecule, for example, when compared to a native unmodified nucleic acid molecule, the overall activity of the modified nucleic acid molecule can be greater than that of the native molecule due to improved stability and/or delivery of the molecule.

In related aspects, the present invention includes use of Locked Nucleic Acids (LNAs) to generate antisense nucleic acids having enhanced affinity and specificity for the target polynucleotide. LNAs are nucleic acid in which the 2′-hydroxyl group is linked to the 3′ or 4′ carbon atom of the sugar ring thereby forming a bicyclic sugar moiety. The linkage is preferably a methelyne (—CH₂-)ₙ group bridging the 2′ oxygen atom and the 4′ carbon atom wherein n is 1 or 2.

Other modifications include 2′-methoxy(2′-O—CH₃), 2′-aminopropoxy(2′-OCH₂CH₂CH₂NH₂), 2′-allyl (2′-CH—CH—CH₂), 2′-O-allyl (2′-O—CH₂—CH—CH₂), 2′-fluoro (2*-F), 2′-amino, 2′-thio, 2′-Omethyl, 2′-methoxymethyl, 2′-propyl, and the like. The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazi-n-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrimido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases are known in the art. Certain of these nucleobases are particularly useful for increasing the binding affinity of the oligomeric compounds described herein. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2 C and are presently preferred base substitutions, even more particularly when combined with 2′-0-methoxyethyl sugar modifications.

Another modification of the antisense oligonucleotides described herein involves chemically linking to the oligonucleotide one or more moieties or conjugates which enhance the activity, cellular distribution or cellular uptake of the oligonucleotide. The antisense oligonucleotides can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Identification of Novel Kinase Regulators of Telomere Length by Pool Screening Approach

Telomere length is maintained at an established equilibrium in the presence of telomerase. Many of the details of telomere length regulation are known in yeast. In humans, many of the players are known, but the molecular details of how length is regulated is not fully understood. Telomere length is regulated at many levels, including transcription, cell cycle regulation, RNA processing, and post translational modifications.

Post-translational modification is a robust method of regulating telomere length. Phosphorylation, ubiquitination, sumoylation, and chromatin modification have all be implicated in telomere length regulation. Understanding these processes is critical for understanding telomere biology, but also provides valuable targets for therapeutic development. Presently, there are few inhibitors targeting telomerase, or known telomere binding proteins. While there have been some attempts at developing telomerase inhibitors, direct inhibition of telomere binding proteins is problematic because they regulate both telomere length and end protection. Loss of telomere binding proteins leads to deprotected telomeres, which are prone to signaling DNA damage or fusing with other telomeres. Kinases are attractive therapeutic targets, given the ease of developing bioavailable, chemical inhibitors against them. Previous work has shown that the kinases ATM (Lee et al., 13 CELL REPORTS 1623-32 (2015)) and CDK1 (Frank et al., 24MOL. CELL 423-32 (2006)) are required for telomere elongation. Thus, the present inventors sought to develop a genetic screen to identify novel kinase regulators of telomere length.

Recent work has demonstrated the power of genetic screens using pooled libraries of shRNAs (Strezoska et al., 7 PLOS ONE e42341 (2012)) or sgRNAs in the presence of Cas9 (Wang et al., 343 SCIENCE 80-84 (2014)). Telomere length can be measured with flow-FISH, which uses hybridization of a fluorescent telomere probe and measuring fluorescence on a flow cytometer (Baerlocher et al., 1 NATL. PROTOC. 2365-76 (2006)). This method was adapted to separate cells based on telomere length using fluorescence activated cell sorting (FACS). While flow-FISH provides single cell accuracy in selecting cells with short or long telomeres, it requires fixing cells. Therefore, the present inventors were unable to use multiple rounds of selection to strengthen selection for long or short telomere cells. The robustness of pooled library screening and the specific selective power of flow-FISH were combined to create a powerful method for identifying novel regulators of telomere length.

### Materials and Methods

shRNA Knockdown.

Expression of hTERT (V3LHS_340163) and POT1 (RHS4430-101129426) was knocked down using pGIPZ lentiviral shRNA vectors (GE Dharmacon). A non-silencing shRNA (GE Dharmacon, RHS4346) was used as a negative control. Lentivirus was generated and cells were transduced. Cells were selected with 2 ig/mL puromycin for 72 hours to eliminate untransduced cells.

gPCR Analysis.

Gene expression of hTERT and POT1 were evaluated 7 weeks post-transduction. Seven weeks is required for sufficient changes in telomere length. qPCR was performed using primers against hTERT and POT1. ARF3 was used as a housekeeping gene to normalize for cell number.

Telomere Flow-FISH Analysis.

Telomere length was determined by flow-FISH with some modifications. Rather than fixation with ethanol, cells were fixed at room temperature in 1.5% paraformaldehyde for 10 minutes, followed by overnight incubation in 100% methanol.

Pooled shRNA Kinase Library.

The Decode Pooled Human GIPZ Kinase Library was used to transduce cells (GE Dharmacon). This is a library of lentiviral particles containing shRNAs, on the pGIPZ vector, against human kinases and kinase related genes. The library contains 4,675 shRNAs against 709 genes. This library can be used in pooled screens, with either positive or negative selection (Strezoska et al., 7 PLoS ONE e42341 (2012)).

Pooled CRISPR Kinase Library.

Pooled CRISPR kinase library was a gift from Eric Lander and David Sabbatini (Addgene #51044). This library is a sub-pool, enriched for kinases, of a larger whole genome CRISPR library (Wang et al., 343 SCIENCE 80-84 (2014)). It contains 5,070 sgRNAs against 507 genes, on the pLX-sgRNA backbone. This backbone does not contain Cas9, which was expressed constitutively from the cell line.

Generating Constitutive Cas9 Expressing Cell Line.

A constitutive Cas9 expressing cell line was generated, and the pooled CRISPR screen was performed. The CRISPR sub-pool backbone contained blasticidin resistance; therefore, a cell line that was blasticidin sensitive was needed. 293FT cells were transduced with lentiCRISPR eGFP V2. This plasmid contained Cas9-FLAG-2a-eGFP in its expression cassette. We sorted transduced cells by FACS (Becton Dickinson) and isolated a population of cells with medium high GFP. Extremely high GFP expression is toxic to cells so a population with moderate-high expression was selected. These cells were counted by haemocytometer, and plated at limiting dilutions in 96-well plates. Individual colonies were isolated, and Cas9 protein level was determined by western blot. The cell line with the highest Cas9 expression level was selected for use in the screen.

Western Blotting.

Protein was isolated by lysis with RIPA buffer (Cell Signaling, #98016S). Protein concentration was determined by BCA analysis (Thermo #23227). Thirty (30) μg of protein was run on a Novex 4-12% Bis-Tris gel (Thermo Scientific) at 200 V for 1 hour, then transferred to a nitrocellulose membrane for 1 hour at 30 V in an XCell II Blot Module (Life Technologies). The membrane was blocked in Odyssey blocking buffer (LI-COR, #927-40000) for 1 hour at RT. The membrane was then incubated in with the following primary antibodies at 4° C.: goat anti-actin (Santa Cruz sc-1616), mouse anti-FLAG (Sigma F3165). The membrane was washed 5 times for 10 minutes with 1×TBST, then cut in half to separate larger proteins where Cas9-FLAG was predicted to run, and smaller proteins where actin was predicted to run. The membranes were then incubated for 2 hours at room temperature in Odyssey blocking Buffer (LI-COR) with the following conjugated secondary antibodies: IRDye® goat anti-mouse 800 for the larger protein membrane, IRDye® donkey anti-goat 680 for the smaller protein membrane (LI-COR). The membranes were then washed 5 times for 10 minutes with 1×TBST, then 2 times for 5 minutes in TBS. Blots were scanned using Odyssey near-infrared scanner (LI-COR).

Transduction and Tissue Culture of Transduced Cells.

Transduction of the both shRNA and CRISPR libraries was carried out at MOI=0.1. Assuming a Poisson distribution of transduction events, an MOI=0.1 will generate >98% of cells with 1 or fewer lentiviral integrations. In the shRNA screen, transduction was performed at 500 fold representation, while in the CRISPR screen, transduction was performed at 100 fold representation. Transduced cells were selected with either 2 pg/mL of puromycin for 3 days, in the case of the shRNA screen, or 10 pg/mL of blasticidin for 7 days, in the case of the CRISPR screen. To ensure fold representation was maintained, cells were cultured such that the population never dropped below the number of originally transduced cells. Cells were cultured, in DMEM media (Gibco) with 10% fetal bovine serum (Gibco) and penstrep glutamine (Gibco), for 7 weeks to allow sufficient telomere length change.

Telomere Flow-FISH Cell Sorting.

Flow-FISH, used for analyzing telomere length (Baerlocher et al., 1 NAT. PROTOC. 2365-76 (2006)), was adapted to sort cells based on telomere length. A large number of cells were required to collect enough genomic DNA to maintain appropriate representation of each integrated shRNA or sgRNA. A number of optimizations were required to enhance flow cytometry efficiency and sorting accuracy to obtain enough cells. First, 4×10⁷ were trypsizinied with 0.05% Trypsin (Gibco) for 5 minutes at 37° C., then washed in PBS (Gibco). Cells were passed through a 70 μM cell strainer (Corning) to eliminate aggregated cells. Cells were pelleted, resuspended in PBS (Gibco) and added dropwise to 1.5% paraformaldeyhyde to a final concentration of 500,000 cells/mL. Cells were incubated with shaking for 10 minutes at room temperature, passed through the cell strainer again, and then resuspended in ice cold methanol at a concentration of 500,000 cells/mL. Cells were incubated for 1-3 weeks at 4° C. in methanol.

To hybridize probe, cells were first passed through a cell strainer, then washed in PBS with 1% BSA (Sigma). Cells were then resuspended in hybridization solution containing FITC telomere probe, at a concentration of 20 million cells/mL, as described (Baerlocher et al., 2006). Probe was hybridized by incubation at 87° C. for 15 minutes, then room temperature overnight.

Cells were washed as described in Baerlocher et al., 2006, and resuspended in modified PI staining solution, at a concentration of 5 million cells/mL. Modified PI staining solution contains PBS with 0.1% Triton X-100, 200 μg/mL RNase A (Sigma), 20 μg/mL propidium iodide (Sigma) and 0.1% SDS. Cells were incubated for 30 minutes, protected from light, before cell sorting.

FACS of Cells by Telomere Length.

Cell sorting was performed on a MoFlo cell sorter as described (Becton Dickenson, 2.3.6). First, single cells were gated on by selecting singlets by pulse width, then gated on G1 based on PI staining, then finally sorting out cells with the longest and shortest 7% of telomere lengths, based on FITC telomere signal. A minimum of 1 million cells were collected in each fraction to collect genomic DNA. A population of unsorted cells were also collected for genomic DNA.

Illumina Sample Preparation and Sequencing.

Genomic DNA was harvested from sorted cells by phenol-chloroform extraction. Phenol chloroform extraction yielded far higher genomic DNA concentration than Puregene DNA extraction (Qiagen), likely due to the harsh formamide treatment during probe hybridization. For shRNA and CRISPR library treated cells, the variable region of the genomic insert was amplified with primers annealing to flanking common regions, as described (Strezoska et al., 2012; Wang et al., 2014). Primers contained indices to allow multiplexing of samples in a single Illumina lane. Amplicons were visualized by gel electrophoresis on a 1% TAE agarose gel, and purified with Agencourt AMPure XP Beads (Beckman Coulter)

PCR products were sequenced on an Illumina HiSeq 2000 to obtain single end 50 base pair reads. Between 3 and 9 samples were multiplexed on a single lane, depending on quantity of PCR product.

shRNA Screen Sequencing Analysis.

Illumina reads from the shRNA screen were analyzed as described (Strezoska et al., 2012). Reads are first parsed by barcode to separate multiplexed samples. Next, they are aligned to a reference genome, of kinase shRNAs, by bowtie2, as described (Langmead and Salzberg, 9 NAT. METHODS 357-59 (2012)). These alignments generated count tables with numbers of reads of each shRNA in each sample. We then used MAGeCK analysis to rank gene enrichment based on the enrichment of individual shRNAs (Li et al., 15 GENOME BIOL. 554 (2014)). MAGeCK analysis was performed in a paired and unpaired manner. In the paired method, MAGeCK was performed on each individual sample, then the mean rank of each gene was determined by averaging the rank of all 3 replicates. In the unpaired method, gene rankings were determined by comparing the mean number of reads in 3 experimental samples, compared to the mean number of reads in 3 control samples. From these rankings genes were manually selected in the long and short telomere fraction to further characterize.

CRISPR Screen Sequencing Analysis.

Illumina reads from the CRISPR screen were analyzed as described (Wang et al., 2016). The method was very similar to the shRNA screen analysis, except MAGeCK software was used to align sgRNA reads to a custom sgRNA reference genome. From this, the software determined read numbers for each sgRNA, then ranked genes based on enrichment, p-value and number of sgRNAs against that gene that were enriched. Like the shRNA analysis, this was also performed in a paired and unpaired manner.

### Results and Discussion

Identifying Cell Line for Pooled Telomere Length Screening.

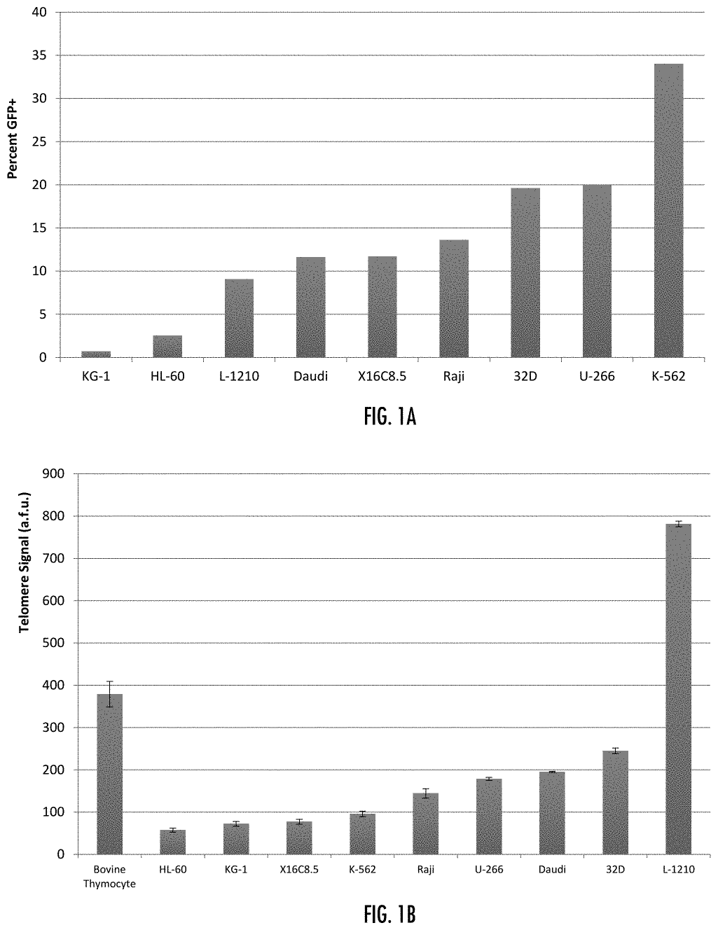

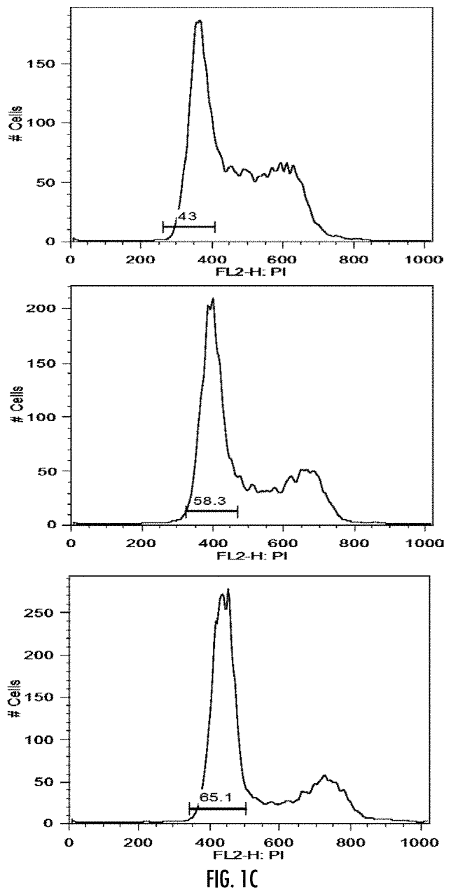

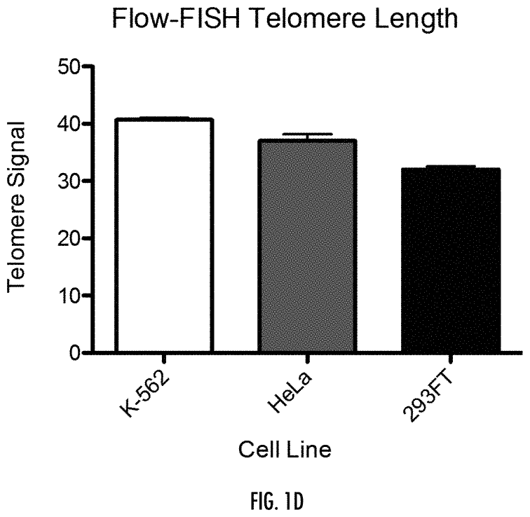

The present inventors sought to identify a cell line in which to perform a pooled screen for novel regulators of telomere length. The present inventors first set out to identify a cell line with moderate telomere length in which dynamic changes in length in either direction could be measured, and that was amenable to FACS. In addition to telomere length, transduction efficiency of multiple cell lines was also measured by transducing of a GFP lentivirus and measuring GFP (FIGS. 1A and 1B). KG-1, HL60, L-1210, Daudi, X16C8.5, Raji, 32D U-266 and K-562 cells were evaluated. K-562 was identified as a candidate cell line due to its high transduction efficiency and moderate telomere length (FIG. 1A, 1B). However, K-562s were not amenable to efficient propidium iodide (PI) staining (FIG. 1C). Flow-FISH requires treating cells with harsh denaturing conditions to hybridize the telomere probe. This had a differential effect on PI staining in different cell lines. PI staining is required for flow-FISH so cells in the G1 phase of the cell cycle can be identified. An accurate cell cycle stain is required to gate on G1 cells because S phase cells and G2 cells will have more telomeres and thus will artificially appear, in this single cell assay, to have longer telomeres. The transduction efficiency for the cells lines with medium telomere length was quite low so other lines were tested. Two other cell lines with high transduction efficiency, 293FT and HeLa, generated more accurate cell cycle profiles (FIG. 1C). They had a medium range of telomere lengths, which were comparable to K-562 (FIG. 1D). Because these cell lines had desired the telomere length, transduction efficiency and cell cycle profile, the present inventors decided to use these cell lines in the screens to identify pathways that, when knocked down, change telomere length.

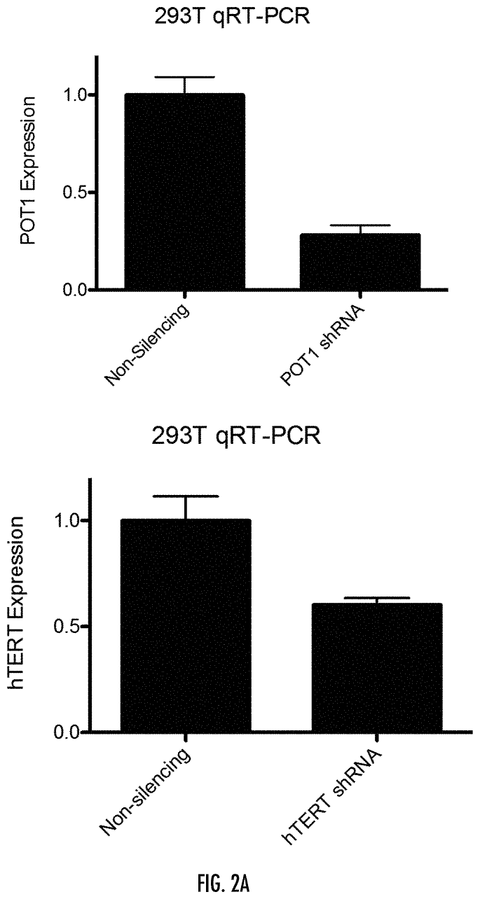

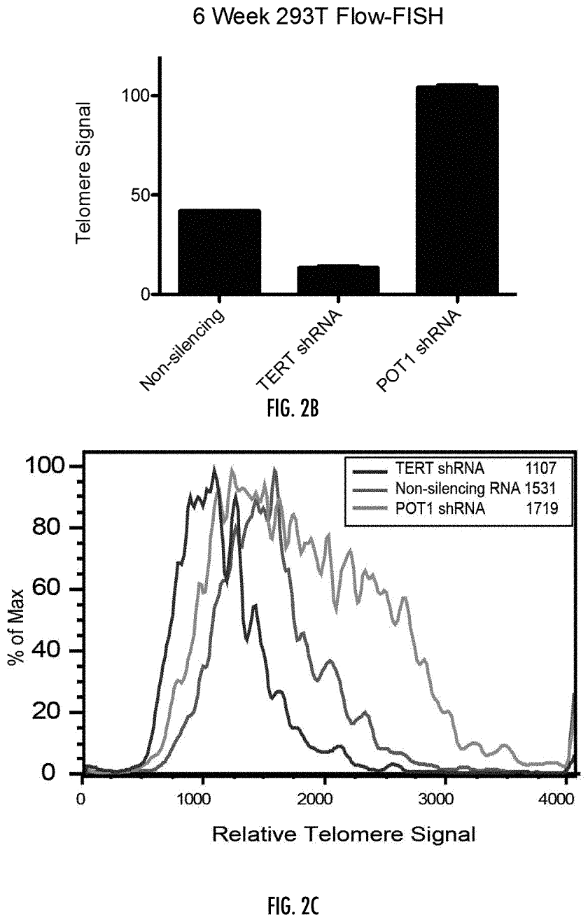

A pilot experiment was conducted to test performance of the screen. Expression of POT1 and hTERT were knocked down by shRNA in separate populations of 293FT cells and cultured for 7 weeks. Previous work has shown that 7 weeks is required to detect changes in telomere length upon POT1 or hTERT knockdown. POT1 knockdown was expected to lengthen telomeres, and hTERT knockdown was expected to shorten them. qPCR indicated that POT1 levels were reduced by approximately 70% and hTERT levels were reduced by approximately 50% (FIG. 2A). hTERT levels are limiting in the cell so even modest knockdown was expected to affect telomere length. Flow-FISH was used to measure telomere length. Consistent with previous reports, hTERT knockdown significantly shortened telomeres, while POT1 knockdown significantly lengthened them (FIG. 2B). Importantly, loss of these known telomere regulators generated a robust change in telomere length. The larger the change in telomere lengths that is generated, the stronger the selective power of the screen will be, indicating the selection strategy could go forward.

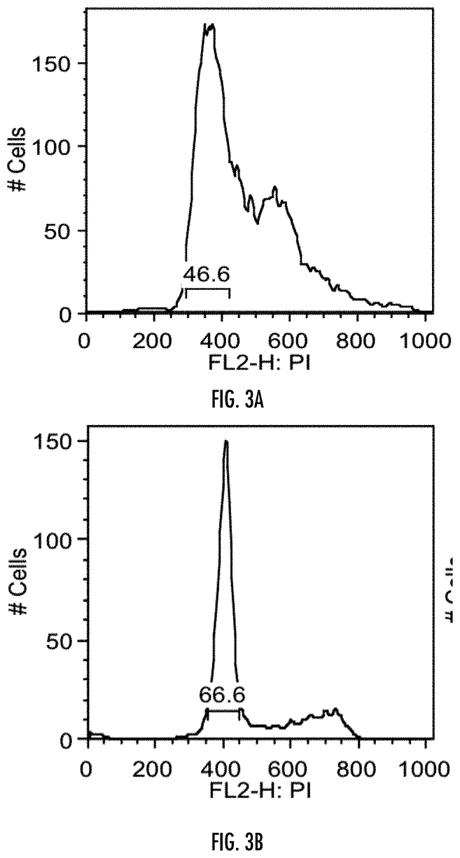

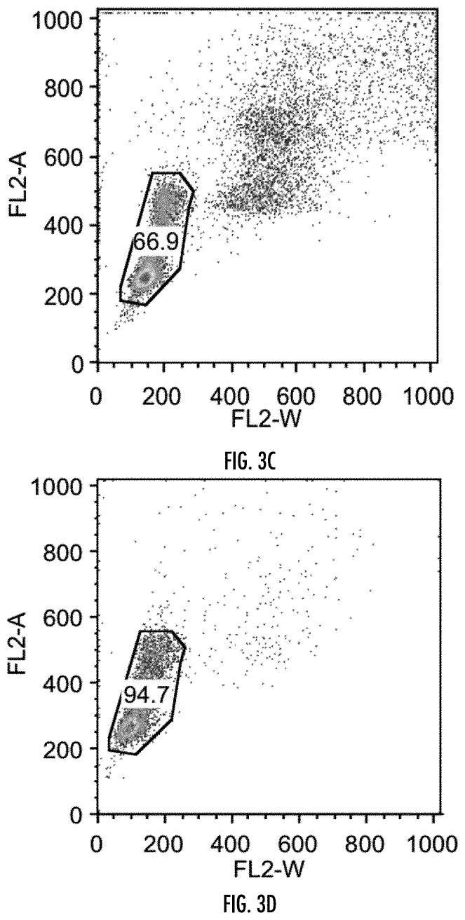

The telomere signals of POT1, hTERT and non-silencing shRNAs were analyzed on the cell sorter and it was noted that loss of POT1 generated long telomeres that did not overlap with the lengths in non-silencing, or hTERT shRNA treated cells (FIG. 3C). Moreover, while hTERT knockdown shifted the telomere signal to the left, compared to non-silencing, the effect was not as dramatic as the long telomere cells (FIG. 3A), presumably because there is a hard limit to how short a telomere can become before a cell undergoes senescence or apoptosis. These data demonstrated that the telomere lengths generated by loss of known regulators could by distinguished and sorted by FACS.

Validating shRNA Screening Approach.

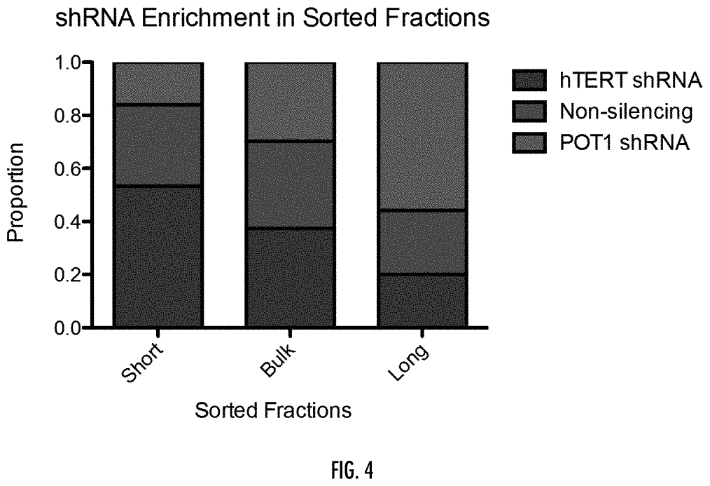

The validity of the screening approach was tested by performing a pilot experiment with known telomere length regulators. The present inventors reasoned that if the screen is working, then it should accurately distinguish known positive and negative regulators of telomere length. In this pilot experiment, a single pool of cells was transduced with equal amounts of hTERT, Non-silencing and POT1 shRNA lentivirus. Loss of hTERT is known to shorten telomeres and loss of POT1 is known to lengthen them. Each of these shRNAs contains a different stem region, but constant regions flanking the stem. After 7 weeks of culture to allow sufficient length change, the 7% longest and shortest telomeres were sorted by flow-FISH. gDNA was collected from the two sorted populations, as well as the unsorted population as a control. From gDNA, the integrated shRNA were amplified from the flanking constant regions. The amplicons were sequenced by Illumina sequencing to determine the relative frequency of cells with hTERT, POT1 or non-silencing shRNA insert in the short, unsorted or long fraction. POT1 shRNA was enriched in the long telomere fraction, and hTERT shRNA was enriched in the short telomere fraction, consistent with the known biology of these genes (FIG. 4A). These data demonstrate that genes with known effects on telomere length are recovered and enriched in the correct population upon flow-FISH sorting and sequencing genomic inserts. These data may underestimate the degree of enrichment because each shRNA was transduced into ⅓ of cells in the population. Because the population is growing competitively, a shRNA that is found in ⅓ of cells to start with can only be enriched by 3 fold as a maximum. However, a shRNA that begins as 1/4675th of a population is capable of enriching significantly more because it comprises a smaller proportion of the initial fraction.

These findings provide confidence that the screening method would allow identification of genes that altered telomere length when knocked down.

Pooled shRNA Screening.

293FT and HeLa cells were used independently to perform a pooled screen for kinase regulators of telomere length. A pooled library of lentivirus containing shRNAs against human protein kinases and some kinase related genes were used (GE Dharmacon). This library has been optimized for pooled screening in a viability screen (Strezoska et al., 2012); it contains 4675 shRNAs directed against 706 kinase and kinase related genes. HeLa and 293FT cells were transduced with 500-fold representation of virus at an MOI of 0.1. This means that, on average, 500 cells were transduced with each shRNA. A high representation is required because the screen uses a relatively weak selection. It also provides additional precision in evaluating more subtle changes in shRNA representation. In this case, only a single round of selection was possible. Successive rounds of enrichment could not be used, for long or short telomeres, because the cells are fixed for flow-FISH and no longer viable. A low MOI was used so that each cell is transduced with 1 or fewer viruses to avoid complications with more than one shRNA interacting in a cell. Transductions were performed in triplicate in both cell lines. These cells were cultured for 7 weeks to allow sufficient changes in telomere length to accumulate in the population.

At 7 weeks, flow-FISH and FACS were performed to sort out cells with the 7% shortest and 7% longest telomeres. Unsorted cells were collected as a control. Many optimizations to the traditional flow-FISH protocol that enhanced the ability to accurately sort cells at a high speed were identified (FIG. 3). Flow-FISH requires formamide treatment of whole cells to allow hybridization of PNA telomere probes. This treatment causes cells to aggregate, even upon cell straining, and resist DNA staining to saturation. It was found that fixing cells with paraformaldehyde, followed by dehydration in methanol produced a far superior cell cycle profile, compared to traditional 70% ethanol fixation (FIG. 3A, 3B). Because a 500 fold representation of ˜5000 shRNAs was required, the present inventors needed to sort approximately 2.5 million cells in the long and short fractions, which required sorting between 200 and 400 million cells total. Thus, faster cell sorting was required to facilitate this. Addition of 0.1% SDS to PI staining solution significantly decreased cell aggregation, allowing for a much faster flow rate (FIG. 3B, 3C).

Because shRNA inserts were integrated into the genome by a lentivirus, genomic DNA was first isolated from sorted populations. Due to flow-FISH treatment, traditional DNA extraction kits (Qiagen), recommended by Illumina, were unable to recover sufficient yields of genomic DNA. Phenol-chloroform extraction provided much higher genomic DNA yield. To amplify shRNA inserts, primers in a flanking common region of the virus were used to amplify the variable stem region that uniquely identifies the shRNAs. These amplified fragments were then purified and sequenced using Illumina deep sequencing to determine the relative frequency of each shRNA insert in the long telomere, short telomere and unsorted populations. Illumina reads were aligned to reference shRNA sequences by bowtie2 (Langmead and Salzberg, 2012), and enriched genes were determined with MAGeCK analysis (Li et al., 2014).

Analysis of Sequencing Results from Pooled shRNA Screen.

Illumina sequencing data were aligned to the reference shRNA sequence library using bowtie2, as described (Strezoska et al., 2012). In this workflow, the number of counts of each shRNA were first determined by aligning to the provided reference library (GE Dharmacon). Next, these counts were normalized based on the total number of reads in a particular sample. This provided a normalized list of reads for each shRNA in the short fraction, long fraction and unsorted fraction, in triplicate, for both HeLa and 293FT screens. MAGeCK analysis (Li et al., 2014) was next used to rank genes associated with the short and long fractions. MAGeCK analysis was originally designed for pooled CRISPR/Cas9 screens, but since it can generate gene rankings from counts tables, the present inventors adapted it for the shRNA screen. The present inventors reasoned that a gene that is involved in telomere length would create a larger enrichment in the long or short fraction, and have multiple shRNAs that target that gene enriched in that particular fraction. MAGeCK uses the p-value, from 3 replicates, and mean enrichment of individual shRNAs to rank genes is likely to be involved in telomere length.

In the HeLa screen, 37 genes enriched in the short fraction and 30 genes in enriched the long fraction were obtained that were statistically significantly (p<0.05). In 293FT, 41 genes enriched in the short fraction and 31 genes enriched in the long fraction were recovered that were statistically significantly (p<0.05). The top 20 genes in long and short fractions for HeLa (Table 1) and 293FT (Table 2) are listed below.

TABLE 1
HeLa shRNA screen top 20 hits

| | | |
|---|---|---|
| Rank | Unpaired | Paired |

A. Short Fraction

| | | |
|---|---|---|
| 1 | GUCY2D | GUCY2D |
| 2 | TEC | PIK3R4 |
| 3 | CDC42BPG | TEC |
| 4 | ANKK1 | CDC42BPG |
| 5 | EIF2AK1 | PIK3R1 |
| 6 | PCK1 | PRKCG |
| 7 | PIK3R4 | CDK6 |
| 8 | TRIO | EIF2AK1 |
| 9 | ILK | ILK |
| 10 | ITPKA | ANKK1 |
| 11 | STK16 | CSK |
| 12 | CSK | PCTK1 |
| 13 | CLK1 | CLK1 |
| 14 | PKN3 | MAP3K11 |
| 15 | PIK3R1 | MASTL |
| 16 | MASTL | STK16 |
| 17 | CAM2N1 | CAMK2N1 |
| 18 | WNK2 | BRD4 |
| 19 | LATS2 | NEK5 |
| 20 | BRD4 | TRIO |

B. Long Fraction

| | | |
|---|---|---|
| 1 | STK16 | ERBB3 |
| 2 | PRKCG | STK16 |
| 3 | TEC | IKBKE |
| 4 | ANKK1 | MAPK14 |
| 5 | RET | TEC |
| 6 | PRAGMIN | PRKCG |
| 7 | PI4KB | PRAGMIN |
| 8 | ITK | LMTK2 |
| 9 | KCNH2 | ANKK1 |
| 10 | IKBKE | ROR1 |
| 11 | MAPK14 | ITK |
| 12 | DGKB | CDC42BPG |
| 13 | CDC42BPG | STK17A |
| 14 | PRKACV | PRKACA |
| 15 | ROR1 | PI4KB |
| 16 | MAP3K15 | PIK3R1 |
| 17 | TAOK1 | MAP3K11 |
| 18 | NEK5 | STK32A |
| 19 | PIK3R1 | ADPGK |
| 20 | PFTK1 | AK7 |

TABLE 2
293 FT shRNA screen top 20 hits

| | | |
|---|---|---|
| Ran | Unpaired | Paired |

A. Short Fraction

| | | |
|---|---|---|
| 1 | MAPK15 | STK10 |
| 2 | TAOK2 | SRMS |
| 3 | CDK3 | PKMYT1 |
| 4 | STK10 | CDKN2DD |
| 5 | PNCK | MAP3K10 |
| 6 | HK2 | PDGFRL |
| 7 | PDGFRL | RPS6KA4 |
| 8 | EPHB2 | AURKB |
| 9 | MAP3K11 | PKN3 |
| 10 | MAPK12 | CALM3 |
| 11 | CDKN2D | CSK |
| 12 | NME3 | TK2 |
| 13 | STK16 | PANK4 |
| 14 | MAPK11 | WNK2 |
| 15 | CSNK1A1 | PIM3 |
| 16 | RELA | STK3 |
| 17 | RPS6KA2 | STK16 |
| 18 | INSRR | STK35 |
| 19 | CALM3 | CDK4 |
| 20 | CDKN1C | TSSK6 |

B. Long Fraction

| | | |
|---|---|---|
| 1 | FES | LIMK1 |
| 2 | DAPK2 | ADCK1 |
| 3 | NUP62 | PI4KA |
| 4 | ADCK1 | CAMK2A |
| 5 | KALRN | KALRN |
| 6 | RPS6KA5 | CDC2L1 |
| 7 | MAPK15 | ACVR1B |
| 8 | PDK4 | DYRK1B |
| 9 | BCR | VRK3 |
| 10 | DYRK1B | TYK2 |
| 11 | PI4KA | INSRR |
| 12 | LIMK1 | LCK |
| 13 | CHEK1 | MAPK15 |
| 14 | IRAK2 | PANK1 |
| 15 | ACVR1B | MAP3K12 |
| 16 | LCK | GRK6 |
| 17 | TK2 | CDK4 |
| 18 | DDR1 | SPHK2 |
| 19 | MYO3B | TSSK6 |
| 20 | CSNK2A2 | EIF2AK2 |

There are relatively few “gold standard” kinases with well-established roles in mammalian telomere length regulation that could be used as positive controls for enrichment. Some positive kinase regulators have been identified, but virtually no negative ones have been described. Therefore, the present inventors were unable to refer to a known kinase regulator of telomere length as a benchmark of enrichment for bona fide telomere length regulators. However, it is noted that the top hit in the 293FT short fraction, MAPK15, was identified, in a previous screen, to lower telomerase activity when it was knocked down (Cerone et al., 71 CANCER RES. 3328-40 (2011)). This demonstrates that the present inventors were able to recover at least one known kinase involved in telomere length regulation.

Upon manual inspection, some genes had significant variation between biological replicates. This was more pronounced in 293FT, than HeLa. This may be explained by the fact that each control is the original unsorted sample from which the long or short fraction was sorted. This control sample controls for varying growth rates and jackpot events in that population, but this could be very different in the other biological replicates. Therefore, each replicate of control, controls best for the experimental replicate that was derived from it. Thus, a paired version of MAGeCK analysis was used to account for this. In this version, MAGeCK was performed on each individual replicate and its corresponding control, and then averaged the ranks of each gene to come up with a final ranking. This analysis yielded significantly different data for the 293FT cells, with nearly half of the top 20 changing, while it did not significantly affect the HeLa cells (Tables 1 and 2). The default MAGeCK analysis were referred to as “unpaired,” and the modified paired version as “paired.” A significant discordance was also found between genes identified by HeLa and 293FT by both paired and unpaired analysis methods. This could be due to differences in cell lines such as mutations altering particular kinase pathways. Another potential source of error is the accuracy of the cell cycle profile. PI staining was used to differentiate G1 cells, so only telomere length within cells with the same DNA content were selected. 293FT cells were significantly more variable than HeLa in the cell cycle staining, suggesting that some of the variability in 293FT may be explained by contaminating S and G2/M cells. Because it showed more consistency between replicates, the HeLa cell line was favored in selecting candidates for further investigation.

The list of candidates to further investigate was determined by comparing their ranking using the paired and unpaired method, exclusivity to one fraction, broad tissue expression and known biological function. Exclusivity to one fraction was used as a criterion because many top hits came up in both the long and short fraction. Loss of telomere regulators is expected to shift mean telomere length, but not alter the breadth of different lengths. Therefore true telomere regulators were expected to be enriched in only the long or short fractions, but not both. Since there were many genes enriched in both fractions, it was concluded that they were likely due to PCR bias and did not represent those genes that broadened telomere length distribution. Instead, the focus was placed on genes that were enriched exclusively in the long or short fraction, but not both. Tissue expression of candidate kinases was determined through the Human Protein Atlas. Broad tissue expression was used as a criterion because telomerase is active in the stem cells of many tissue types. Therefore, genes which were important in regulating telomere length would be expressed in a broad array of tissues. For example, a gene which is expressed exclusively in neurons is unlikely to have a global role in telomere length regulation. To determine biological function, candidates were manually inspected and their function was evaluated through published literature. Genes with nuclear localization and association with known telomere pathways such as DNA damage, cell cycle regulation, checkpoint regulation, DNA replication and chromatin modification were favored.

Pooled CRISPR Screening Approach.

Having established the best methods to carry out the flow-FISH screen, these methods were used to test the next generation of genome editing libraries. shRNA libraries were very successful in past screens. CRISPR libraries recently became available and might offer a complementary approach to identify genes involved in telomere length regulation. A pooled CRISPR screen was performed to complement the pooled shRNA screening approach. CRISPR has the advantage that it will completely knock out a gene by disrupting the genomic locus. On the other hand, shRNA can have a variable level of knock down, depending on the shRNA. This can be advantageous if the gene is essential because a partial loss will produce a viable phenotype. Alternatively, CRISPR produces gene knockouts, and enrichment in a population may be due to complete loss rather than partial loss of function. Pooled CRISPR screens have been used successfully to demonstrate the feasibility this approach (Shalem et al., 343 SCIENCE 84-87 (2014); Wang et al., 2014). In addition, libraries have been developed that are enriched for sgRNAs against particular groups of genes, including kinases. A kinase CRISPR sub pool containing 5070 sgRNAs against 507 human kinases (Wang et al., 2014) was used.

A cell line of 293FT cells which constitutively expressed Cas9 was generated. 293FT was chosen because this line had the most dynamic range of telomere lengths based on previous experiments (FIG. 2B). This cell line was generated by transducing 293FT cells with lentiCRISPR V2 eGFP, a lentiviral vector encoding Cas9-2a-eGFP. Cells with high eGFP expression were then sorted, and clones were isolated by serial dilution. Western blotting of 6 clones with high GFP expression were used to select the one with the highest Cas9 expression.

The CRISPR screen was performed in a similar manner to the shRNA screen. It was carried out in triplicate in the Cas9-eGFP 293FT cell line. This screen was performed at 100-fold representation of sgRNAs because fewer sgRNAs were expected to be enriched since essential genes would be expected drop out entirely if they were homozygously deleted. 100 and even 20 fold representation of pooled screens have been shown to produce the same results as 500 fold representation screens (Strezoska et al., 2012). The lower representation allowed us to sort the shortest and longest 6% of cells instead of 7%, which provided more stringency in selection. Primers specific to flanking regions of the sgRNA were used to amplify sgRNA inserts from genomic DNA. PCR products were sequenced by Illumina sequencing and enrichment was determined using MAGeCK analysis (Li et al., 2014) as described below.

Analysis of Sequencing Results from Pooled CRISPR Screen.

CRISPR screen results were analyzed in a similar manner to the shRNA screen. MAGeCK contains alignment algorithms as well, the present inventors were able to directly analyze FASTQ files from Illumina sequencing, rather than first align with bowtie2. Paired and unpaired analysis were performed. The top 20 hits from the short and long, MAGeCK analysis are listed in Table 3.

TABLE 3
CRISPR screen top 20 hits

| | | |
|---|---|---|
| Rank | Unpaired | Paired |

A. Short Fraction

| | | |
|---|---|---|
| 1 | HIPK2 | MAPK10 |
| 2 | PIK3R4 | HIPK2 |
| 3 | TBK1 | PIK3R4 |
| 4 | CAMK2B | FGFR4 |
| 5 | GRK7 | GRK7 |
| 6 | MAPK8 | MAPK8 |
| 7 | DCLK1 | TSSK3 |
| 8 | PRKD2 | PKMYT1 |
| 9 | TAOK2 | CDC42BPB |
| 10 | STK3 | GRK4 |
| 11 | AATK | SMG1 |
| 12 | NEK6 | MAST2 |
| 13 | RIOK1 | CDK13 |
| 14 | PRKCB | RIOK1 |
| 15 | FLT4 | AATK |
| 16 | SMG1 | CSNK1E |
| 17 | MAPK10 | KIT |
| 18 | CDC42BPB | PRKD2 |
| 19 | PKMYT1 | DMPK |
| 20 | BRAF | CDKL1 |

B. Long Fraction

| | | |
|---|---|---|
| 1 | NEK4 | NEK9 |
| 2 | FLT4 | CSNK1A1L |
| 3 | CSN1A1L | PRKCE |
| 4 | GUCY2C | IGF1R |
| 5 | PIM2 | FLT4 |
| 6 | GRK1 | NEK3 |
| 7 | MAPK15 | TSSK6 |
| 8 | PRKCE | ZAK |
| 9 | LRRK2 | PRKG2 |
| 10 | PRKG2 | MAP4K5 |
| 11 | EIF2AK3 | GRK7 |
| 12 | NEK9 | PIK3R4 |
| 13 | TYRO3 | NEK10 |
| 14 | OBSCN | TBK1 |
| 15 | TSSK4 | TGFBR2 |
| 16 | GRK7 | NEK11 |
| 17 | MAP3K9 | PIM2 |
| 18 | NEK10 | MINK1 |
| 19 | MKNK2 | TEK |
| 20 | ADRBK2 | ROCK1 |

Less concordance was seen between biological replicates compared to the shRNA screen. This may be explained by the lower 100-fold representation that was chosen, compared to 500-fold in the shRNA. Furthermore, CRISPR relies on NHEJ of the genomic locus to knockout all alleles of a particular gene. Since 293FT cells have variable ploidy, it is possible that some cells did not have homozygous deletions. This would lead to an effective lower representation, because only a fraction of transduced cells would knockout the gene.

The CRISPR screen also had poor concordance with the 293FT and HeLa shRNA screen. Only one short fraction hit out of the top 20 was shared with HeLa short, and 1 long fraction hit out of the top 20 was shared with 293FT long.

Candidates for further validation were chosen in the same manner as in the shRNA screen, by evaluating ranking in paired and unpaired analysis, exclusive enrichment in one fraction, broad tissue expression and known biological function. Genes that were shared between the shRNA and CRISPR screen were given the highest priority. Candidates which had not been tested from the shRNA screen were then selected based on availability of drug inhibitors, known function and broad tissue distribution.

### SUMMARY

The present inventors have developed a versatile method for identifying novel genes that regulate telomere length. Cells were treated with pooled lentiviral libraries of either shRNA or sgRNAs, and cells were then sorted based on telomere length by telomere flow-FISH. shRNA or sgRNA inserts in these cells were amplified and sequenced by Illumina sequencing. By comparing frequency of shRNA or sgRNA reads in the long or short fraction, compared to the unsorted fraction, putative negative and positive regulators of telomere length were defined.

While there are limitations to this assay, particularly the relatively week selective power of flow-FISH, it is highly versatile. Multiple rounds of culture and flow-FISH for long or short telomere cells could not be performed because flow-FISH requires fixing cells. Here, the present inventors focused on knocking out or knocking down kinases. However, this method can be used for other kinds of libraries, or even the whole genome. This provides a powerful method to discover more novel telomere length regulators in future experiments.

TABLE 4
Primer List

| | |
|---|---|
| Primer | Sequence |

Pooled shRNA Screen

| | |
|---|---|
| Decode Forward | AATGATACGGCGACCACCGAGATCTACACCGGTGCCTGA |
| PCR Primer | GTTTGTTTGAA (SEQ ID NO: 1) |
| Decode Reverse | CAAGCAGAAGACGGCATACGAGATGGCATTAAAGCAGC |
| PCR Primer | GTATCCAC (SEQ ID NO: 2) |
| Decode Illumina | GAAGGCTCGAGAAGGTATATTGCTGTTGACAGTGAGCG |

Sequencing Primer (SEQ ID NO: 3)
Pooled CRISPR Screen

| | |
|---|---|
| Sequencing Primer | CGGTGCCACTTTTTCAAGTTGATAACGGACTAGCCTTATT |
| | TTAACTTGCTATTTCTAGCTCTAAAAC (SEQ ID NO: 4) |
| Indexing Primer | TTTCAAGTTACGGTAAGCATATGATAGTCCATTTTAAAA |
| | CATAATTTTAAAACTGCAAACTACCCAAGAAA (SEQ ID NO: 5) |
| Reverse Primer | AATGATACGGCGACCACCGAGATCTACACCGACTCGGTG |
| | CCACTTTT (SEQ ID NO: 6) |
| Forward Primer 1 | CAAGCAGAAGACGGCATACGAGATCATCACGTTTCTTGG |
| | GTAGTTTGCAGTTTT (SEQ ID NO: 7) |
| Forward Primer 2 | CAAGCAGAAGACGGCATACGAGATCCGATGTTTTCTTGG |
| | GTAGTTTGCAGTTTT (SEQ ID NO: 8) |
| Forward Primer 3 | CAAGCAGAAGACGGCATACGAGATCTTAGGCTTTCTTGG |
| | GTAGTTTGCAGTTTT (SEQ ID NO: 9) |
| Forward Primer 4 | CAAGCAGAAGACGGCATACGAGATCTGACCATTTCTTGG |
| | GTAGTTTGCAGTTTT (SEQ ID NO: 10) |
| Forward Primer 5 | CAAGCAGAAGACGGCATACGAGATCACAGTGTTTCTTGG |
| | GTAGTTTGCAGTTTT (SEQ ID NO: 11) |
| Forward Primer 6 | CAAGCAGAAGACGGCATACGAGATCGCCAATTTTCTTGG |
| | GTAGTTTGCAGTTTT (SEQ ID NO: 12) |
| Forward Primer 7 | CAAGCAGAAGACGGCATACGAGATCCAGATCTTTCTTGG |
| | GTAGTTTGCAGTTTT (SEQ ID NO: 13) |
| Forward Primer 8 | CAAGCAGAAGACGGCATACGAGATCACTTGATTTCTTGG |
| | GTAGTTTGCAGTTTT (SEQ ID NO: 14) |
| Forward Primer 9 | CAAGCAGAAGACGGCATACGAGATCGATCAGTTTCTTGG |
| | GTAGTTTGCAGTTTT (SEQ ID NO: 15) |
| Forward Primer 10 | CAAGCAGAAGACGGCATACGAGATCTAGCTTTTTCTTGG |
| | GTAGTTTGCAGTTTT (SEQ ID NO: 16) |
| Forward Primer 11 | CAAGCAGAAGACGGCATACGAGATCGGCTACTTTCTTGG |
| | GTAGTTTGCAGTTTT (SEQ ID NO: 17) |
| Forward Primer 12 | CAAGCAGAAGACGGCATACGAGATCCTTGTATTTCTTGG |
| | GTAGTTTGCAGTTTT (SEQ ID NO: 18) |

### Example 2: Chemical Inhibition of BRD4, CK1 and MEK/ERK Block Telomere Elongation

Replicative immortality is a hallmark of cancer. Most adult mammalian cells do not express telomerase, and will eventually undergo replicative senescence as telomeres shorten. One of the most common ways cancer cells overcome this is by activating telomerase expression. Telomerase is able to maintain telomeres such that they are not recognized as DNA damage, and the cell is able to divide indefinitely. Thus, developing telomerase inhibitors, or inhibitors of telomere elongation, are an area of significant investigation. Here, the present inventors identify BRD4, CK1 and the MEK/ERK pathway as positive regulators of telomere length. Identification of these pathways generates new potential targets for blocking telomere elongation in cancer.

BRD4 is a BET family protein. BET family proteins are characterized by bromodomains, which bind to acetylated lysines on histones. BRD4 has both histone acetyl transferase activity, and kinase activity. BRD4 has previously been found to be required for the growth of acute myeloid leukemia (Zuber et al., 478 NATURE 524-28 (2011)) and other cancers (French et al., 63 CANCER RES. 304-07 (2003)). Many BRD4 inhibitors have entered various stages of clinical trials (Boi et al., 21 CLIN. CANCER RES. 1628-38 (2015)); NCT02259114; NCT01587703; NCT02308761; NCT01949883; NCT02157636. Despite its therapeutic consideration, relatively little is known about BRD4's mechanism of action. BRD4 inhibition blocks MYC transcription (Mertz et al., 108 PROC. NATL. ACAD. SCI. USA 16669-73 (2011)), but this is unlikely to account for all of its effect, since it is more efficacious than traditional MYC inhibitors.

BRD4 has pleiotropic roles associated with its role as a scaffold, kinase and histone acetyl transferase (Devaiah et al., 100 J. LEUKOC. BIOL. 679-86 (2016)). BRD4 acts as a mitotic “bookmark,” by acting as a scaffold for transcription factors on promoters of genes to be expressed after mitosis (Dey et al., 463 MOL. BIOL. CELL 4899-4909 (2009)); Zhao et al., 13 NAT. CELL. BIOL. 1295-1304 (2011)). It also directly regulates transcription by recruiting pTEFb (Yang et al., 19 MOL. CELL 535-45 (2005)) and phosphorylating the C-terminal domain of RNA Polymerase II (Devaiah et al., 109 PROC. NATL. ACAD. SCI. USA 6927-32 (2012)). BRD4 uses its histone acetyl transferase activity to evict nucleosomes from chromatin, through acetylation of H3 and (Devaiah et al., 23 NAT. STRUCT. MOL. BIOL. 540-48 (2016)). It has also been found to affect cell cycle progression, by regulating Aurora Kinase B (You et al., 29 MOL. CELL. BIOL. 5904-5103 (2009)).

In addition to BRD4, the present inventors found the inhibition of CK1 and MEK/ERK, blocked telomere elongation. The CK1 family of kinases has diverse cellular roles including DNA repair, proliferation, cytoskeleton regulation, trafficking, apoptosis, and differentiation (Knippschild et al., 4 FRONT. ONCOL. 96 (2014)). It is broadly expressed in mammalian tissues, but has multiple isoforms with distinct regulatory roles (Knippschild et al., 17 CELL SIGNAL 675-89 (2005)). It has been shown to activate p53 during stress or DNA damage (Dumaz et al., 463 FEBS LETT. 312-16 (1999)). CK1 also regulates diverse pathways including Wnt, Hippo and Hedgehog (Knippschild et al., 2014). It is an active target of research in cancer, partially due to its role in regulating p53, but also its role in the aforementioned pathways. Identifying isoform specific roles and developing isoform specific drugs, also remains an active area of investigation.

The MAPK kinase pathway is a well-studied and diverse signaling pathway, which communicates extracellular information to the cell. Ligand binding to a cell surface receptor activates Ras, which activates Raf, which phosphorylates MEK, which phosphorylates ERK (Chin et al., 400 NATURE 468-72 (1999)). This activates a variety downstream effects related to proliferation, differentiation and inflammation. Constitutive activation of the MAPK is a common source of unregulated proliferation in cancer (Osborne et al., 22 CELL. RES. 14-22 (2012)). Many inhibitors targeting this pathway are in various stages of clinical trial, or already being used to treat cancer. The MAPK pathway has also been found to regulate telomerase transcription (Ge et al., 26 MOL. CELL. BIOL. 230-37 (2006)) and expression levels of telomere binding proteins (Picco et al., 58 ANTIMICROB. AGENTS CHEMOTHER. 1501-15 (2016)).

Here, the present inventors identify BRD4, CK1 and MEK/ERK as candidate positive regulators of telomere length.

### Materials and Methods

Small Molecule Inhibitors.

Small molecule inhibitors were dissolved in DMSO, then added to cells at indicated concentrations. Small molecules are listed in Table 5.

TABLE 5
Small Molecule Inhibitors

| | | Catalogue | |
|---|---|---|---|
| Drug | Vendor | Number | Target |
| KU-55933 | R&D Systems | 3544 | ATM |
| JQ1 | Selleckchem | S7110 | BRD4 |
| OTX015 | Selleckchem | S7360 | BRD4 |
| I-BET151 | Selleckchem | S2780 | BRD4 |
| MS436 | Selleckchem | S7305 | BRD4 |
| D4476 | Selleckchem | S7642 | CK1 |
| 5-Iodotubercidin | Selleckchem | S8314 | CK1 |
| IC261 | Selleckchem | S8237 | CK1 |
| GDC-0994 | Selleckchem | S7554 | ERK1/2 |
| FR-180204 | Selleckchem | S7524 | ERK1/2 |
| Trametinib | Selleckchem | S2673 | MEK1/2 |
| Selumetinib | Selleckchem | S8314 | MEK1/2, ERK1/2 |

Virus Production and Titering.

SVA is a lentiviral vector containing mTR, driven by its endogenous promoter, and mTERT-IRES-eGFP, driven by the PGK promoter. To generate SVA lentivirus, 15-cm polystyrene plates (BD Falcon) were first coated with 10 mL of 100 μg/ml poly-D lysine for 30 minutes. Next 8 million 293FT cells were plated in DMEM media (Gibco) with 10% fetal bovine serum (Gibco) and penicillin-streptomycin-glutamine (Gibco). After 24 hours, media was changed to 1% FBS in DMEM and SVA mTR (mTR lentiviral vector), pCMVA8.91 (containing gag and pol lentiviral genes), and VSVG (containing the env lentiviral gene) were co-transfected. Transduction was performed with Lipofectamine 2000 (Life Technologies) in Opti-MEM media (Gibco). Supernatant was collected after 48 hours, briefly centrifuged to remove cellular debris and filtered through a 0.45 m CN filter (Thermo) to eliminate remaining debris. Viral supernatant was concentrated using 100 kDa ultracentrifugal filters (Millipore) and centrifuging for 10 minutes at 4000g. Concentrated virus was then collected, aliquoted and frozen at −80° C.

To titer virus, 10⁵ 293FT cells were seeded and cultured overnight. A doubling time of 24 hours was assumed, yielding a population of 2×10⁵ cells at the time of transduction. Cells were treated with 8 μg/ml of polybrene (Sigma) and infected with 1, 2, 4 and 8 LL of virus. Media was changed 24 hours post transduction and cells were collected at 48 hours post transduction. The percentage of GFP-positive cells was determined by flow cytometry (Becton Dickinson). From these data, percent GFP-positive were converted to initial transduction units by multiplying percent GFP by 2×10⁵, the initial number of cells at time of transduction. Transduction units were then plotted versus volume added, and a line of best fit was generated across the linear range of data points. The slope of this line represented the titer of the virus.

Inhibition of SVA Mediated Telomere Elongation.

First, 2×10⁵ SL13 cells were plated onto a 6-well dish in DMEM (Gibco) with 10% FBS (Sigma) and 1% Penicillin-Streptomycin-Glutamine (Gibco). SL13 cells are a mouse fibroblast line that was derived as described (Lee et al., 299(Pt 1) BIOHCEM. J. 123-28 (2015)). After 24 hours, the drug or vehicle was added at the indicated concentration for the duration of the experiment. 24 hours later, SVA virus was added at an MOI of 0.5. 2 population doublings were estimated during the initial 48 hours; therefore 400,000 transduction units of SVA were added to 800,000 cells. Media was changed at 24 hours post transduction, and cell pellets were collected at 2 and 6 days post transduction.

Telomere Southern Blot.

Telomere length was measured by telomere restriction fragment Southern blot. Genomic DNA was extracted using Puregene Core Kit A (Qiagen). 1-3 pLg of gDNA was digested with Msel (NEB) and loaded onto a 0.7% TAE Agarose gel. The gel was run at 37 V for 16 hours, then denatured for 30 minutes in 0.5 M NaOH and 1.5 M NaCl, then neutralized for 30 minutes in 1.5 M NaCl and 0.5 M Tris-HCl pH 7.4. The DNA was transferred from the gel to a nylon membrane (Amersham Hybond N+) by the weighted method overnight. Next, DNA was crosslinked to the membrane with a UV Stratalinker (Stratagene). The membrane was then pre-incubated for 2 hours at 65° C. with Church's Buffer. Telomere probe and 2-log ladder (Life Technologies) was then end-labeled with Klenow fragment polymerase (NEB) 33 μM of dATP, dTTP, dGTP and 50 μCi of a-32P dCTP (3000 Ci/mmol). Unincorporated nucleotides were removed by running on a G50 column (GE Healthcare). Labeled probe was counted, denatured at 100° C., and 10⁶ counts/mL telomere probe and 2×10⁴ counts/mL of 2-log ladder were added. The membrane was probed overnight at 65° C. The next day, the membrane was washed twice with 2×SSC 0.1% SDS, then twice with 0.5×SSC 0.1 SDS. The membrane was exposed on a phosphorimager (GE Healthcare) and imaged on a STORM scanner (GE Healthcare).

### Results and Discussion

Telomeres are Rapidly Elongated by Telomerase Overexpression.

The present inventors sought to validate shRNA and CRISPR screen results with an independent assay. Knocking out candidate genes by CRISPR/Cas9 and evaluating telomere length was initially considered. Telomere length changes typically take greater than 6 weeks to detect by Southern blot. Thus, this method would limit the number of genes that could be evaluated, particularly as a first pass to validate screen results. The present inventors decided to evaluate candidates based upon their ability to block telomere elongation upon chemical inhibition. This assay has been previously used to demonstrate the requirement of ATM for telomere elongation (Lee et al., 2015). In this assay, mouse fibroblasts are transduced with a SVA, a telomerase lentivirus, which overexpresses telomerase. In the presence of vehicle alone, SVA causes robust telomere elongation after just 6 days. However, upon chemical inhibition of a protein such as ATM, which is required for telomere elongation, no telomere elongation is observed.

While this assay is limited to evaluating candidates enriched in the short fraction of the screen, it is robust, fast and generates a clear answer. Furthermore, partial elongation blockage could be detected by observing telomeres with partial elongation. An additional benefit of this assay is that telomerase is overexpressed from a randomly integrated lentivirus, so its transcription is unaffected by factors that regulate endogenous telomerase transcription. Many proteins have been previously found to affect TERT transcription. This assay exclusively detects proteins that regulate telomere length at the post-transcriptional level.

The present inventors have previously observed that this assay is best suited to testing chemical inhibitors, rather than siRNA knockdown. This may be due to the significant telomerase overexpression, which may overcome knockdown of a positive regulator by mass action. Chemical inhibitors, however, can be present at much higher concentrations in the cell, and provide much greater inhibition of target proteins, compared to siRNA. Because the screen focused on kinases, many of the candidates had well-established chemical inhibitors, which were used to validate the candidate telomere length regulators.

BRD4 Inhibition Blocks Telomere Elongation.

BRD4 was identified as a potential positive regulator of telomere length in our shRNA screen. It is a bromo-domain containing protein with histone acetyl transferase activity, as well as kinase activity. It is known to regulate gene transcription, by modifying chromatin at promoters, or acting as a scaffold for additional transcription factors. It has also been found to regulate phosphorylation of the C-terminal domain of RNA Polymerase II.

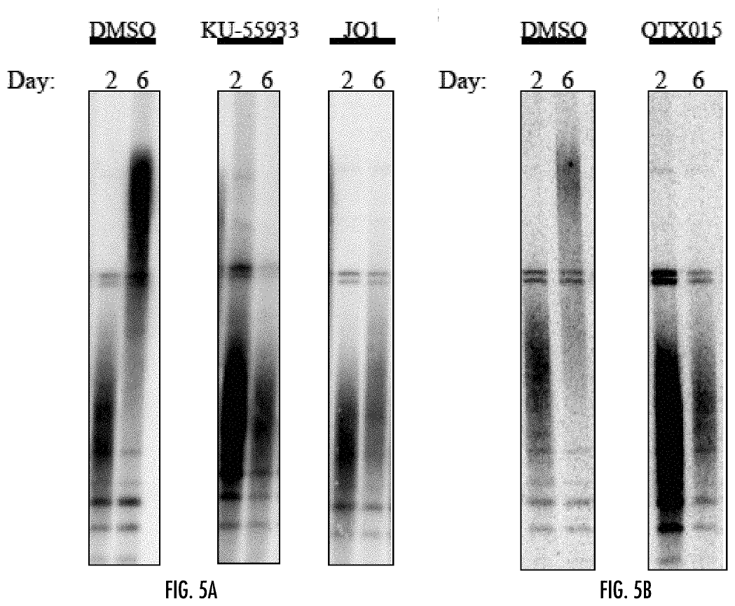

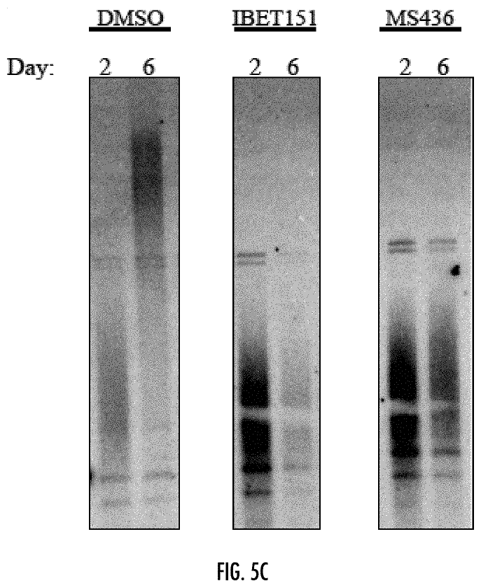

Inhibition of BRD4 with 0.1 μM JQ1, a well-established BRD4 inhibitor, blocked telomere elongation by SVA (FIG. 5A). Telomere length 6 days after transduction with SVA was similar to day 2, indicating that BRD4 inhibition blocked telomere elongation in this context. The magnitude of this blockage was similar to the result seen with 10 μM KU-55933, a potent ATM inhibitor (FIG. 5A). Additional BRD4 inhibitors were examined next, including OTX015, I-BET151 and MS436, and these inhibitors also blocked SVA induced telomere elongation (FIG. 5B, 5C). Since inhibition was seen with four independent BRD4 inhibitors, it is likely that the blocking of telomere elongation is in fact due to BRD4 inhibition rather than off target effect.

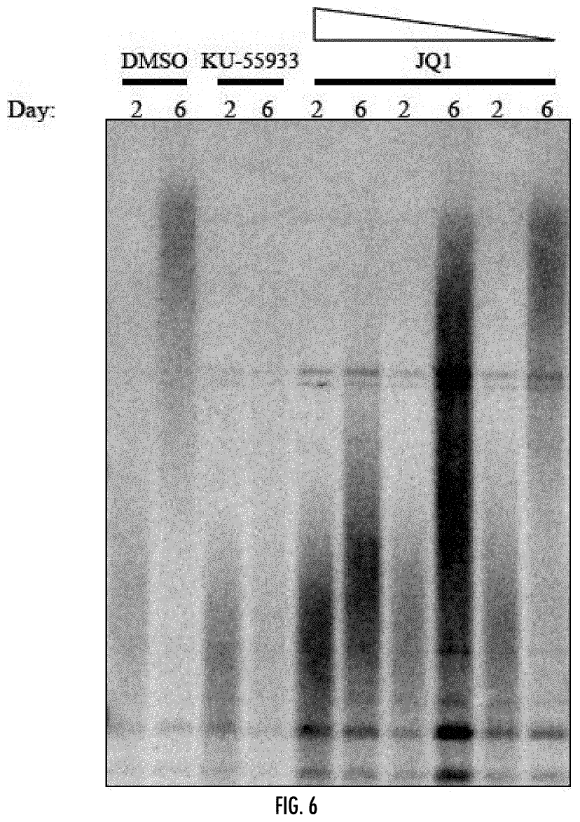

To examine whether JQ1 inhibition of telomere elongation was dose dependent, several concentration of this drug were tested. As previously observed, 100 nM JQ1 blocked nearly all SVA mediated elongation (FIG. 6). However, 11 nM JQ1 had virtually no effect on telomere elongation, while 33 nM JQ1 produced an intermediate telomere length. This demonstrates that JQ1's effect on blocking telomere elongation titrates with its concentration. This is further underscores the conclusion that inhibiting BRD4 is what is responsible for blocking telomere elongation.

CK1 Inhibition Blocks Telomere Elongation.

CK1 was identified as potential positive regulators of telomere length in the pooled CRISPR screen. The effect of chemically inhibiting this kinase on telomere elongation by SVA was tested. Three different CK1 inhibitors: D4476, 5-Iodotubercidin and IC261 were used. KU-55933, an ATM inhibitor, was used as a positive control for blocking telomere elongation.

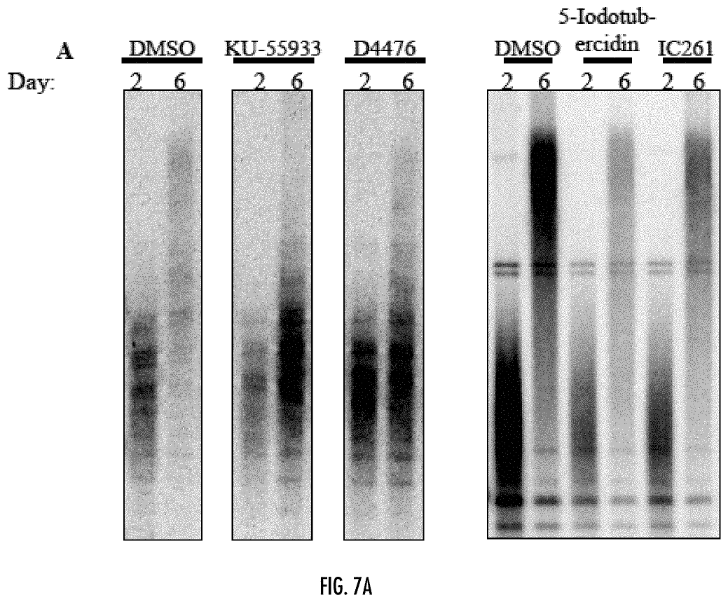

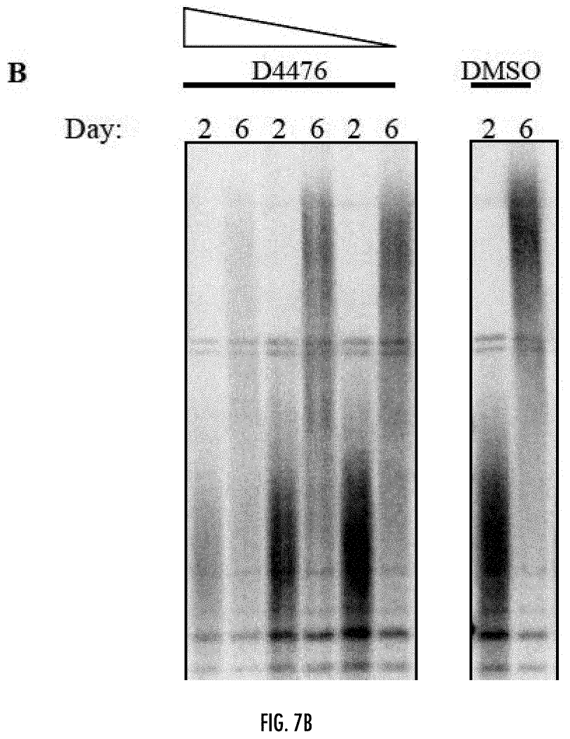

Inhibition of CK1 by 10 μM D4476 blocked telomere elongation (FIG. 7A), to a comparable degree as 10 μM KU-55933, an ATM inhibitor, which is known to inhibit telomere elongation (Lee et al., 2015). This indicated that loss of CK1 strongly inhibited telomere elongation. However, 0.5 μM 5-Iodotubercidin or 0.5 μM IC261, which are also CK1 inhibitors, did not affect telomere elongation (FIG. 7A). Therefore, either the effect seen with D4476 was an off target effect, or 5-Iodotubercidin and IC261 did not block CK1 as strongly as D4476 did. The present inventors observed that D4476's effect was concentration dependent, as lower concentrations of D4476 caused less blockage of telomere elongation (FIG. 7B). Notably, even 0.37 μM D4476 was able to block some telomere elongation, compared to DMSO control. These data indicates that CK1 may be a potential positive regulator of telomere length.

Because only 1 out of 3 CK1 inhibitors tested blocked telomere elongation, it is not definitive that CK1 is required for telomere elongation. However, this assay shows that CK1 inhibition is sufficient to block telomere elongation. It may be that the difference observed between different inhibitors is due to variable degree of CK1 inhibition. Firstly, 5-Iodotubercidin is not a specific CK1 inhibitor. It was originally identified as an adenosine kinase inhibitor (Phillis and Smith-Barbour, 53 LIFE SCI. 497-502 (1993)). It was only later identified to affect CK1 (Massillon et al., 299(Pt. 1) BIOCHEM. J. 123-28 (1994)), however, the off target effects were broad, and its inhibition of CK1 has not been characterized or validated. D4476 is a more potent inhibitor of CK1 than IC261, with an in vitro IC50 of 200 nM, compared to an IC50 of 16 μM for IC261 (Selleckchem). IC261 was also more toxic to mouse fibroblasts than D4476, as 2.5 μM IC261 caused significant cell death, while D4476 was tolerated at nearly 50 μM (data not shown). Given the higher in vitro IC50 and lower tolerated in vivo concentration, it is may be that that IC261 caused less CK1 inhibition than D4476 in this assay. D4476 has been shown to be a superior CK1 inhibitor in vivo using a variety of downstream effects (Bryja et al., 120 J. CELL SCI. 586-95 (2007); Rachidi et al., 58 ANTIMICROB. AGENTS CHEMOTHER. 1501-15 (2014); and Rena et al., 5 EMBO REPORTS 60-65 (2004)). D4476 also inhibits all CK1 isoforms while IC261 only targets some (Rena et al., 2004), which may point to telomere specific isoforms of CK1. Given these data, it may be that the greater telomere elongation blockage of D4476 is due to its greater specificity for Ck1 inhibition.

MEK1/2 and ERK1/2 Inhibition Blocks Telomere Elongation.

A number of components of the MAPK pathway were identified that affected telomere elongation in our pooled shRNA and CRISPR screens. MEK1/2 and ERK1/2 are two critical, and well-studied components of the Ras-Raf-MEK-ERK pathway. The present inventors sought to determine the effect of inhibiting MEK1/2 or ERK1/2 on telomere elongation by SVA.

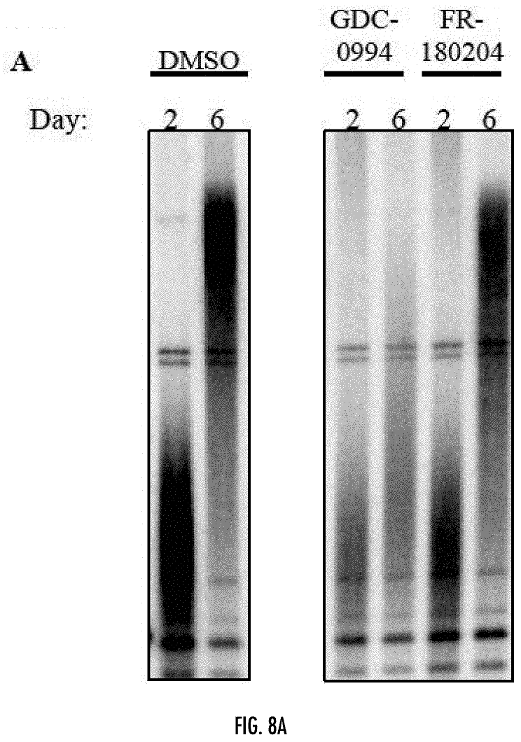

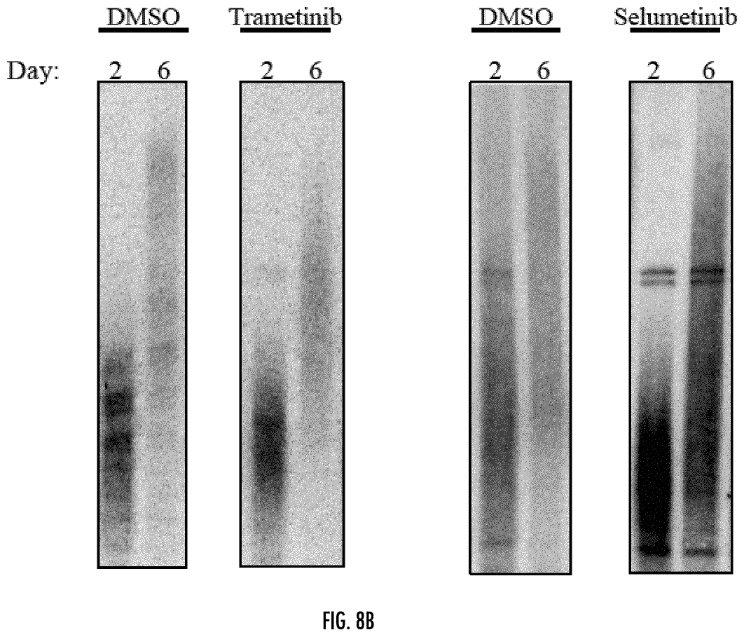

Specific inhibition of ERK1/2 by 10 μM GDC-0994 blocked telomere elongation (FIG. 8A). However, treatment with 10 μM FR-180204, another ERK1/2 specific inhibitor, had minimal effect on telomere elongation compared to DMSO control (FIG. 8A). Treatment with 10 nM Trametinib, a MEK1/2 specific inhibitor, partially blocked telomere elongation, as did treatment with 5 iM selumetinib, a MEK1/2 and ERK1/2 inhibitor.

These data indicate that the MEK/ERK pathway is a positive regulator of telomere elongation. However, the specific mechanism of action is still unclear. The effect on telomere elongation is not due to changes in telomerase transcription since telomerase was expressed from a lentivirus and driven by a constitutively active promoter. Inhibition of ERK1/2 alone by GDC-0994 caused the most significant blockage of telomere elongation; although FR-180204, another ERK1/2 inhibitor, had minimal effect on elongation (FIG. 8A). This effect was slightly weaker than that observed from BRD4, CK1 or ATM inhibition. Inhibition of MEK1/2 alone was sufficient to moderately block elongation (FIG. 8B). Inhibition of MEK1/2 and ERK1/2 together also moderately blocked telomere elongation (FIG. 8B). There are two potential explanations for why different effects on telomere elongation were observed with drugs targeting the same pathway: some drugs may have off target effects, which are causing or preventing the telomere elongation phenotype, or some drugs are not effectively inhibiting the target. The degree to which MEK or ERK was inhibited by the drug has not been assessed. Therefore, variable kinase inhibition by each drug cannot be ruled out. However, in this case all drugs used have been demonstrated as potent and specific inhibitors of their intended target in vitro (Selleckchem). An alternative explanation would be that each drug has a different effect on downstream effectors, which are the direct regulators of telomere elongation. For example, RSK and the NFκB pathways are downstream of MEK/ERK (Carter and Hunninghake, 275 J. BIOL. CHEM. 27858-64 (2000); and Roux et al., 282 J. BIOL. CHEM. 14056-64 (2007)). The present inventors identified elements of both pathways, which may be downstream effectors of the MEK/ERK pathway that are directly regulating telomere length

### SUMMARY

The present inventors provided evidence that chemical inhibition of BRD4, CK1 and MEK/ERK block telomere elongation in a telomerase overexpression assay. 4 independent BRD4 inhibitors were identified that all blocked telomere elongation. In addition, it was found that 1/3 CK1 inhibitors blocked telomere elongation. Those that were not effective could be due to poor CK1 inhibition. Finally, inhibition of ERK1/2, MEK1/2 or both blocked telomere elongation although the effect was weaker than BRD4 or CK1. These results may be due to either variable inhibition by different drugs, or variable inhibition of a yet-to-be identified downstream effector in the pathway.

Further work is required to determine if these drugs can block telomere elongation by endogenous telomerase. Moreover, identifying the mechanism of these targets may facilitate the design of more potent and specific inhibitors.

These data suggest that BRD4, CK1 and the MEK/ERK pathways may be therapeutic targets for telomerase inactivation in cancer. Some BRD4 inhibitors are already in clinical trials as cancer drugs, and many ERK/MEK pathway inhibitors are approved for clinical use. Elucidating the mechanism by which these targets block telomere elongation will allow clinicians to better target cancers or other indications where inhibiting aberrant telomere elongation has therapeutic benefit.

1. A method of treating cancer in a subject by interfering with lengthening of telomeres in cancer cells, comprising administering to the cells an effective amount of an inhibitor of casein kinase 1 (CK1), wherein the administration of the inhibitor leads to progressive telomere shortening in the cancer cell, thereby treating cancer in the subject.

2. The method of claim 1, wherein said cancer is selected from the group consisting of stomach cancer, osteosarcoma, lung cancer, pancreatic cancer, adrenocortical carcinoma, melanoma, breast cancer, ovarian cancer, cervical cancer, skin cancer, connective tissue cancer, uterine cancer, anogenital cancer, central nervous system cancers, retinal cancer, blood and lymphoid cancers, kidney cancer, bladder cancer, colon cancer and prostate cancer.

3. The method of claim 1, wherein the inhibitor is a small molecule, a peptide, a nucleic acid molecule, or a protein.

4. The method of claim 3, wherein the nucleic acid molecule is an siRNA, shRNA, miRNA, Locked Nucleic Acid (LNA), antisense oligonucleotide, a chemically modified oligonucleotide, or a combination thereof.

5. The method of claim 1, further comprising administering to the cells an effective amount of an inhibitor of bromodomain-containing protein 4 (BRD4) and/or an inhibitor of the MEK/ERK pathway.

6. The method of claim 1, further comprising administering a chemotherapeutic agent.

7. The method of claim 1, wherein the subject is human.

8. The method of claim 1, further comprising measuring telomere length.

9. The method of claim 8, wherein telomere length is measured prior to and after contacting with the inhibitor.

10. The method of claim 8, wherein measuring comprises flow cytometry combined with fluorescent in situ hybridization (flow-FISH).

11. The method of claim 8, wherein the measuring comprises a Southern blot.

12. The method of claim 8, wherein the measuring comprises a modified single telomere length analysis (STELA).

13. A method of increasing telomere length comprising contacting a cell with an agent that activates BRD4, CK1, MEK/ERK pathway or a combination thereof, thereby elongating telomeres in the cell.

14. The method of claim 13, wherein the agent is a small molecule, a peptide, a nucleic acid molecule, or a protein.

15. The method of claim 14, wherein the nucleic acid molecule is an siRNA, shRNA, miRNA, Locked Nucleic Acid (LNA), antisense oligonucleotide, a chemically modified oligonucleotide, or a combination thereof.

16. The method of claim 13, wherein the agent increases expression or activity of BRD4.

17. The method of claim 13, wherein the agent increases expression or activity of CK1.

18. The method of claim 13 wherein the agent is an activator of the MEK/ERK pathway.

19. The method of claim 13, wherein the cell is a mammalian cell.

20. The method of claim 19, wherein the cell is a human or murine cell.

21. The method of claim 13, wherein telomere length is increased at least 50% as compared to telomere length prior to contacting with the agent.

22. The method of claim 13, further comprising measuring telomere length.

23. The method of claim 22, wherein telomere length is measured prior to and after contacting with the agent.

24. The method of claim 22, wherein measuring comprises flow-FISH.

25. The method of claim 22, wherein the measuring comprises a Southern blot.

26. The method of claim 22, wherein the measuring comprises STELA.

27. A method of treating a telomere syndrome in a subject, wherein the syndrome is characterized by shortened telomere length, the method comprising administering to the subject a BRD4 agonist, a CK1 agonist, a MEK/ERK pathway agonist or a combination thereof, wherein administration of the agonist leads to progressive telomere lengthening, thereby treating the telomere syndrome in the subject.

28. The method of claim 27, wherein the syndrome is selected from dyskeritosis congenita, bone marrow failure, aplastic anemia, and pulmonary fibrosis.

29. The method of claim 27, wherein the agent is a small molecule, a peptide, a nucleic acid molecule, or a protein.

30. The method of claim 27, wherein the CK1 agonist is pyrvinium paomoate.

31. The method of claim 27, wherein the subject is human.

32. The method of claim 27, wherein telomere length is increased at least 50% as compared to telomere length prior to contacting with the agonist.

33. The method of claim 27, further comprising measuring telomere length.

34. The method of claim 33, wherein telomere length is measured prior to, and after contacting with the agonist.

35. The method of claim 33, wherein measuring comprises flow-FISH.

36. The method of claim 33, wherein the measuring comprises a Southern blot.

37. The method of claim 33, wherein the measuring comprises STELA.

38-74. (canceled)
